(19) United States
(12) Patent Application Publication
(10) Pub. No.: US 2011/0269920 A1
(43) Pub. Date: Nov. 3, 2011

(54) FUNCTIONAL POLYMERS AND NOVEL COMPOSITES FOR CO2 SEQUESTRATION AND RELEASING OF FERTILIZER CONVERSION, CO2 FOAMING, AND THEIR APPLICATIONS

(75) Inventors: Yong G. Min, New Albany, OH (US); James L. Lee, Columbus, OH (US)

(73) Assignee: NANOMATERIAL INNOVATION LTD., Columbus, OH (US)

(21) Appl. No.: 13/106,673

(22) Filed: May 12, 2011

Related U.S. Application Data
(63) Continuation of application No. 13/096,661, filed on Apr. 28, 2011.
(60) Provisional application No. 61/343,374, filed on Apr. 28, 2010.

(51) Int. Cl. C08G 73/02; B01J 20/26; C07C 211/55; B82Y 30/00

(52) U.S. Cl. 525/540; 564/434; 502/402; 977/700; 977/742; 977/762

(57) ABSTRACT

The present invention discloses a CO₂ reservoir. The CO₂ reservoir comprises a functional conducting polymer and a plurality of particles. The particles are coated with the functional conducting polymer, and the particles comprise nanoscale or microscale particles and their mixture.

### CROSS REFERENCE TO RELATED APPLICATIONS

This application is a continuation of U.S. Ser. No. 13/096,661, filed Apr. 28, 2011, which claims the benefit of U.S. Provisional Application No. 61/343,374 filed Apr. 28, 2010.

### BACKGROUND OF THE INVENTION

1. Field of the Invention

The present invention relates to the synthesis of functional polyanilines (FPANs) and preparation of their composites for CO₂ sequestration and by-product (particularly fertilizers) conversion and CO₂ based polymeric foams, and methods of their production and articles made from them.

2. Description of the Prior Art

The ongoing human-induced emission of carbon dioxide (CO₂) threatens to change the earth's climate, i.e., global warming. For this invention, families of novel polymers and their composite particles were developed for CO₂ absorption and capitulation. The objective was to use functional polymers, such as polyaniline (PAN or Pani)—the most stable and low cost conducting polymer with high CO₂ affinity, to form functional polymers such as functional polyaniline (FPAN), and then use FPAN and/or FPAN modified particles such as montmorillonite nanoclay (MMT), graphite, Boron Nitride (BN), and activated carbon as additives to form novel composite particles. The conducting polymer, along with their dopants, are not only coated on the surface of particles, but also intercalated into the particles. These novel functional polymers and their composite particles can act as a “molecular CO₂ reservoir” to control the CO₂ absorption and release. They can be used as a CO₂ collecting material to remove CO₂ from air, and fillers in polymeric materials for CO₂ based foaming. As CO₂ absorption media, these particles have the potential to be used directly in the absorption systems including fluidized beds, packed bed and membrane reactors. Fast CO₂ release can be achieved through various methods including vacuum pumping and an acid-base vapor treatment process. The by-product or end-products can be dry ice, which can be used as cooling media or stored underground; H₂CO₃ acid for industrial use; CaCO₃ and Ca(HCO₃)₂ as industrial raw materials; or CO₂ containing fertilizers such as NH₄HCO₃, (NH₄)₂CO₃, KHCO₃, K₂CO₃, NH₄HSO₄ (from SOₓ) NH₄NO₃ (from NOₓ), etc. In addition, we can also use naturally available inorganic particles from miners and volcanic ash to conduct the treatment reaction to generate fertilizers such as, Apatite, Ca-silicate, Feldspar Episodes, etc. Through a dual-chemical loop design, the aforementioned CO₂ sequestration and by-product (particularly fertilizers) conversion process can be carried out continuously under harsh conditions as in an electric power plant.

Polymer foams have been widely used for thermal insulation, and other construction, wind energy transportation and packaging applications. Due to environmental concerns of the currently used blowing agents—chlorofluorocarbons (HCFCs) and fluorocarbons (HFCs)—carbon dioxide (CO₂) has been identified as one of the most promising foaming agents for polymer foams because it is nonflammable, inexpensive, nontoxic and environmentally friendly. However, CO₂ also has its drawbacks of low solubility and high diffusivity in polymers compared to existing CFC/HCFC/HFC blowing agents. This often results in inferior foam density and cell morphology. As a result, the foam insulation performance is often low and inconsistent. Pani-particle nanocomposites act as a “molecular CO₂ reservoir” to adsorb and control CO₂ release during foaming, allowing cost effective replacement of CFC/HCFC/HFC blowing agents.

### SUMMARY OF THE INVENTION

In view of the foregoing, it is an object of the embodiment of the present invention to provide a CO₂ reservoir for CO₂ sequestration and conversion to useful by-products using a dual chemical loop design. The materials can also be used in the polymer foaming processes.

According to one embodiment, a CO₂ reservoir is disclosed. The CO₂ reservoir comprises a functional conducting polymer and a plurality of particles. The particles are coated with the functional conducting polymer, and the particles comprise nanoscale or microscale particles and their mixture.

According to another embodiment, a method of CO₂ capture and conversion comprises loop I and loop II. In the loop I, CO₂ is by absorbed the CO₂ reservoir to form doped FPAN—HCO₃₋. In the loop II, NH₃/H₂O is absorbed by the FPAN—HCO₃₋ to form de-doped FPAN—NH₄₊.

### BRIEF DESCRIPTION OF THE DRAWINGS

The present disclosure can be described by the embodiments given below. It is understood, however, that the embodiments below are not necessarily limitations to the present disclosure, but are used to a typical implementation of the invention.

FIG. 1: (I) lists of the chemical synthesis process of CO₂ functional polymers, where A containing at lest one of functional groups, which is CO₂ affinity function; and B containing at least one of functional groups, which is NH3 affinity function; (II) lists of the chemical synthesis process of functional polyaniline (FPAN), which containing at least one of CO₂ affinity groups and at least one of NH3 affinity groups; (III) an example of FPAN, which has a CO₂ affinity group of —OH, and a NH3 affinity group of —COOH, (IV) an example of FPAN, which has a CO₂ affinity group of —OH, and a NH₃ affinity group of —SO₃H; (V) an example of functional ethylene copolymer, which has a CO₂ affinity group of —OH, and a NH3 affinity group of —COOH.

FIG. 2A: Highly branched/side-chain polyaniline and their associating compounds (doping) and pH range control for CO₂ absorption/releasing. In the polyaniline structure, the R would be amine and hydroxyl (—OH) groups, where the amine will act as chemical association to the CO₂, and the hydroxyl group will act as physical association to the CO₂.

FIG. 2B: The proposed mechanism of CO₂ sequestration and NH₄HCO₃ fertilizer formation.

FIG. 3A: The open circle potential (OCP) measurements vs. reaction time during the in-situ polymerization. The “diamond dots” represents the in-situ polymerization of pure aniline (sample Pani-HCl from Table 1 bath-1). The “square dots” represents the in-situ polymerization of aniline with MMT (sample MMT-HCl bath-1 from Table 1 control).

FIG. 3B: Open circle potential (OCP) measurements vs. reaction time during the in-situ polymerization.

FIG. 4: conductivity vs. pH curve from (a) PAN—HCl, (b) PAN-PSSA/DBSA, (c) Self-doped PAN-1, (d) Self-doped PAN-2, (e) PAN-PSSA-Graphene.

FIG. 5A: A schematic of the in-situ polymerization of aniline with MMT to form (A) polyaniline nanoclay composite at doped Emeraldine Salt (ES) form (PAN-ES-MMT), and (B) de-doped Emeraldine Base (EB) form (PAN-EB-MMT).

FIG. 5B: A schematic of the in-situ polymerization of aniline with dispersed graphene.

FIG. 5C: Functional polymer/particle composites and associating with CO₂. In the polyaniline structure, the R would be amine and hydroxyl (—OH) groups, where amine acts as chemical association to CO₂, and the hydroxyl group acts as physical association to CO₂.

FIG. 5D: In-situ polymerization of aniline in graphite leading to expended graphite.

FIG. 5E: Conducting polymer/particle composites and associating with CO₂.

FIG. 6: XRD measurements on PAN-MMT nanocomposites with various inorganic/organic acids/salts as dopants, where the MMT-Na is the virgin grade of MMT clay from the vendor, MMT-ES-PSSA is PSSA doped PAN-MMT, and MMT-PSSA2-ES is another batch of PSSA doped PAN-MMT composites, where the MMT/Aniline ratio is listed in Table 1.

FIG. 7: Lab set-ups to test solid and liquid samples on CO₂ absorption and releasing rate, and testing the reuse treatment and byproducts, where (1) N₂ tank, (2) CO₂ tank, the flow rate is controllable. Typically, we used 15% CO₂ mix with 85% of N₂, (3) the humidity and temperature control unit, (4) the sample holder, the solid composite sample is placed in and capped with glass woods, (5)CO₂ by-product reactor and testing for liquid samples, (6) a heater with a magnetic stirring, (7) a syringe for volume measurement, (8) a weight to adjust the mechanical resistance of the syringe, (9) the Rubotherm high pressure absorption instrument, testing CO₂ absorption and release at both vacuum and high pressure (up to 150 bar) in both solid and liquid samples.

FIG. 8A: Schematic flow chart of novel composite working hypothesis on CO₂ absorption/release and production of industrial chemicals.

FIG. 8B: Chemical reactions of polyaniline (PAN) with CO₂ absorption and release and conversion to fertilizers, where (i) is PAN-EB/CO₂ absorption reaction, (ii) is PAN-ES/CO₂ release reaction with NH₄OH and NH₄HCO₃ fertilizer is generated as a by-product, (iii) is PAN-ES/CO₂ release reaction with KOH and KHCO₃ fertilizer is generated as a by-product, (iv) PAN-ES/CO₂ release reaction with Zn(OH)₂ and ZnCO₃ fertilizer is generated as a by-product.

FIG. 8C: Weight changes at three different medium systems at 4M solution concentration.

FIG. 8D: FTIR studies of concentrated solution after NH₄OH de-doping process.

FIG. 9A: Schematic illustration of CO₂ sequestration and conversion to fertilizers.

FIG. 9B. Schematic process diagram of CO₂ sequestration and conversion to fertilizer, the left process (I) for the solid sorbent and the right process (II) for the liquid sorbent.

FIG. 9C: Schematic illustration of a continuous belt design of CO₂ sequestration process, where the CO₂ active material is coated on a substrate (glass mat as a example) and the substrate moves like a transition belt to absorb the CO₂ from any CO₂ rich area, and release the CO₂ through NH₄OH washing. The washed solution is then dried to form fertilizer, and the regenerated substrate can be reused for next cycle.

FIG. 10A: XRD analysis results on polyaniline nanoclay composites and polystyrene blends with different dopants at both doped form (ES) and de-doped form (EB).

FIG. 10B: Electric resistance measurements of FPAN—CO₂—NH₃ reaction in gas phase showing very fast reaction rate.

FIG. 10C: UV/vis spectrum and color changes of FPAN—CO₂—NH₃ reaction in water showing very fast reaction rate (˜10 seconds per cycle), where the “blue color” film is FPAN in-situ polymerized on PET film under the treatment of 0.1 M NH₄OH solution with pH value of 9, and the “green color” film is the same film under the treatment of CO₂/H₂O solution with pH value of 5.

FIG. 11A: SEM micrographs (×100 magnification) of PS filled with (a) HCl-doped PAN-MMT composites, (b) de-doped HCl-PAN-MMT composites, (c) pTSA-doped PAN-MMT composites, (d) de-doped pTSA-PAN-MMT composites; (e) MgSO₄—HCl doped PAN-MMT composites, (f) de-doped MgSO₄—PAN-MMT composites. The left 3 SEM pictures show an open-cell structure, and the right 3 SEM pictures show a closed-cell structure.

FIG. 11B shows the thermal stability of FPAN vs. MEA in regard to CO₂ sequestration at 60 and 80° C.

FIGS. 12A-12F: SEM micrographs (×100 magnification) of PS filled with (a) DBSA-doped PAN-MMT composites, (b) de-doped DBSA-PAN-MMT composites, (c) PSSA-doped PAN-MMT composites, (d) de-doped PSSA-PAN-MMT composites; (e) PSSA-N+R4 doped PAN-MMT composites, (f) de-doped PSSA-N+R4-PAN-MMT composites.

FIG. 12G: XRD measurements on PAN-MMT nanocomposites with various inorganic/organic acids/salts as dopants.

FIG. 12H: XRD analysis results on polyaniline nanoclay composites and polystyrene blends with different dopants at both doped form (ES) and de-doped form (EB).

FIG. 13: CO₂ absorption test results. (a) 5 wt. % of polyaniline disposed graphite or graphene filled PS blend and (b) the same amount graphite filled PS blend. The CO2 foaming condition was at 125° C./2000 psi for 24 hours. It took about 120 seconds to open the pressure chamber. Therefore, there were almost 150 seconds delay to record the first data in the 4-digital balance. There was also some temperature fluctuation during the test. In order to avoid those errors, an accuracy test was conducted using Rubotherm instrument. (c) Sample-A CO₂ absorption was about 11.73 g CO₂/100 g sample at 70° C., and (d) Sample-B CO₂ absorption was about 7.28 CO₂/100 g sample at 70° C.

FIG. 14: The ratio of UV/vis absorption intensity at different wavelengths with the UV/vis absorption intensity for polystyrene (PS at ˜280 nm). The dispersed GF samples showed the lowest absorption comparing to pure PS and OC's commercial materials (OC and OC-new). The low color could be due to the conducting polymer intercalation into the graphite (dispersed graphene) and polystyrene intercalating with the interacted particles.

FIG. 15: SEM micrographs (×100 magnification) of PS filled with (a) HCl-doped PAN-MMT composites, (b) de-doped HCl-PAN-MMT composites, (c) pTSA-doped PAN-MMT composites, (d) de-doped pTSA-PAN-MMT composites; (e) MgSO₄—HCl doped PAN-MMT composites, (f) de-doped MgSO₄—PAN-MMT composites.

FIG. 16: SEM micrographs (×100 magnification) of PS filled with (a) DBSA-doped PAN-MMT composites, (b) de-doped DBSA-PAN-MMT composites, (c) PSSA-doped PAN-MMT composites, (d) de-doped PSSA-PAN-MMT composites; (e) PSSA-N+R4 doped PAN-MMT composites, (f) de-doped PSSA-N+R4-PAN-MMT composites.

Table 1: Data on synthesis of PAN-MMT nanocomposite particles with different dopants.

Table 2: Data on synthesis of PAN-graphite/graphene composite particles with different dopants.

Table 3a: Data on CO₂ absorption for PAN-inorganic/organic composites solid particles. Table 3b indicates that conducting polymer can significantly enhance CO₂ absorption. We propose that the conducting polymer acts as a catalyst to reduce the entropy of CO₂ associating to the amines; therefore, more CO₂ can be quickly associated to the polymeric backbones and/or particles (on both surface and intercalation) chemically and/or physically.

Table 3b: Data on CO₂ absorption for PAN-inorganic/organic composites liquid samples. Table 3c indicates that the conducting polymer associated polyionic/polycationic (or polyelectrolyte) can significantly enhance the CO₂ absorption. We propose that the conducting polymer acts as a catalyst to reduce the entropy of CO₂ associating to the amines, therefore, more CO₂ can be quickly associated to the polymeric backbones (in solution) and/or particles chemically and/or physically.

Table 3c: Conversion of captured CO₂ to NH₄HCO₃ for liquid and solid sorbents.

Table 3d: Elemental analysis results of CO₂ regenerated NH₄HCO₃.

Table 4: Summary of XRD for PAN-MMT nanocomposites and PAN-MMT-PS blended polymers.

Table 5: Density and cell density of PAN-MMT-PS foams.

Table 6: UV absorption ratios from UV-vis spectrum of PS foam samples.

### DESCRIPTION OF THE PREFERRED EMBODIMENTS

What probed into the invention is a method of quickly capture CO₂ and convert it into useful by-products. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

The present invention discloses low-cost, thermally stable and high CO₂ affinity conducting polymers and their association compounds (i.e., dopants) as a “molecular CO₂ reservoir” to not only achieve effective CO₂ capture under harsh conditions as in an electric power plant but also convert the sequestered CO₂ into useful products such as NH₄HCO₃ fertilizer in a simple reaction and allow the “molecular CO₂ reservoir” to be re-used in a continuous process.

The new functionalized polyaniline composite particles can also be blended with various polymers to generate polymer foams different cell morphologies by utilizing CO₂ as a blowing agent because they could act as a “molecular CO₂ reservoir” to adsorb and control CO₂ release during foaming. As an example, the acid doped polyaniline could generate open cells, which should be valuable for acoustic and filtration applications. However, the salt de-doped polyaniline composite would generate small cell size and low foam density, which should be valuable for thermal insulation applications.

The present invention relates to the synthesis of composites using functional polymers and their associated compounds—coated particles in particulate-like, plate-like or fiber-like form with high CO₂ and water affinity. Typical particles are clay, silica, alumina, TiO₂, Talc, Boron Nitride (BN), graphite, graphene, carbon nanotubes, carbon nanofibers, active carbons, carbon woods, carbon black, carbon fiber, glass fiber, glass beads, zeolite, polymeric beads, polymeric particles, etc. Typical polymer coatings are conducting polymers, such as polyaniline, polypyrrole, polythiophene, polyacetylene, polyphenylene, polyphenylene vinylene, their derivatives and copolymers, as well as self-doped conducting polymers (the dopants act as a side chain attached to the main chain of polymer). In general, people use “doping” and/or “dopant” for conducting polymer, i.e., a small amount additives that enhance the conductivity. According to this present invention, “associating compounds” can be used to broadly cover the concept of “dopant”, since the “associating compound” will not only enhance the conductivity, but also enhance other properties including CO₂ absorption and release. The associating compounds are used to associate the functional polymers as well as particles to enhance composite performance in terms of conductivity, charges, wettability, CO₂ absorption, water, solvent, small molecular absorption, hydrophilicity and hydrophobicity, positive and negative charges, etc. Based on their categories and functions, the associating compounds can be classified into the following different types: (1) “p-type” associating compounds that will supply positive charge to the functional polymer; (2) “n-type” associating compounds that will supply negative charge to functional polymers; (3) protonation associating compounds that will supply protons to functional polymers; (4) ionic associating compounds that will supply ions to functional polymers; (5) ionic liquid associating compounds that will supply ionic liquid to functional polymers, (6) polymeric associating compounds that will supply polymeric charges to functional polymers; (7) surfactant associating compounds that will supply surfactants to conducting polymers, etc. The above functional polymers with their associating compounds are coated onto the above particles through various processes such as in-situ polymerization, vapor phase polymerization, solution coating, spray coating, solution blending, thermal blending and ink-jetting, etc. These functional polymers and their coated particles can be used as CO₂ collecting material to remove CO₂ from air and condense it into dry ice or react it with other species into useful products.

These composite particles can be further blended with both thermoplastic and thermoset polymers (such as polystyrene, PMMA, nylon, PET, PP, TPO, PVC, PEEK, PU, PA, PI, PEI, PLA, PCL, liquid crystal polymers, epoxy, phenolics, etc.) to form a minor phase with high CO₂ solubility and water affinity through solution blending, thermal extrusion, compounding, injection molding processes, etc. In addition, the presence of these composites can change the color of the coated particles, e.g. turning dark black graphite particles into a lighter color due to inter-composite dispersion among the functional polymers, associating compounds and particles, and intra-composite dispersion between the polymer matrix and composites. These polymer or polymer blend composites are then used to produce high-performance foam products by extrusion, injection molding, batch foaming, etc. for insulation and structural applications such as thermal insulation, fire resistance, compression resistance, CO₂ absorption, air filtration, anti-static, and EMI shielding, etc.

Synthesis of functional conducting polymers such as polyaniline, polypyrrole, polythiophene, polyphenylene vinylene, polyphenylene, their derivatives and functional conducting polymer coated nanoscale and microscale particles as a “molecular CO₂ reservoir” to not only achieve high-speed CO₂ capture under harsh conditions as in an electric power plant but also convert the sequestered CO₂ into useful products such as NH₄HCO₃ fertilizer in a simple reaction and allow the “molecular CO₂ reservoir” to be re-used in a continuous dual chemical process.

Accordingly, a CO₂ reservoir is disclosed in this invention. The CO₂ reservoir comprises a functional conducting polymer and a plurality of particles coated with the functional conducting polymer. The particles comprise nanoscale or microscale particles and their mixture. The functional conducting polymer comprises CO₂ affinity group and NH₃ affinity group. The functional conducting polymer comprises one or any combination selected from the group consisting of polyaniline, polypyrrole, polythiophene, polyphenylene vinylene, polyphenylene and their derivatives.

One preferred embodiment according to this specification discloses a CO₂ reservoir with a functional conducting polymer, and the functional conducting polymer is

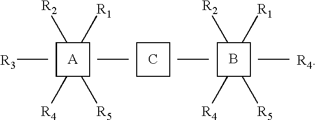

The method of forming the functional conducting polymer according to this embodiment comprises the following formula (I):

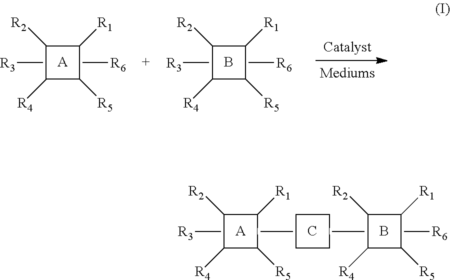

wherein A is selected from one of the following: —H, —CH₂—, —CH₂—CH₂—, —(CH₂)ₓ—, —C₆H₆—, C₆H₁₀—, —C₁₀H₆—, —C₁₄H₈—, —C₁₈H₁₂, —CH═CH—, —C═C—, etc., wherein B is selected from one of the following: —H, —CH₂—, —CH₂—CH₂—, —(CH₂)ₓ—, —C₆H₆—, C₆H₁₀—, —C₁₀H₆—, —C₁₈H₁₂—, —CH═CH—, —C═C—, etc., wherein , the binding group C is selected from one of the following group: —CH₂—, —CH₂—CH₂—, —(CH₂)ₓ—, —C₆H₆—, —C₆H₄—, —C₆H₁₀—, —C₁₀H₆—, —C₁₄H₈—, —C₁₈H¹²—, —CH═CH—, —C═C—, —NH—, —N═, —O—, —CO—, —COO—, —CONH—, —S—, —SO—, —SO₂—, —PO₂—, —P═N—, —BH—, —B(OH)—, wherein R₁˜R₂ are individually selected from one of the following CO₂ affinity groups: —NH₂—, —(R)ₓ—NH₂— wherein R is —(CH₂)ₓ—, —(CH₂)ₓ—CO—NH₂, —B(OH)₂, —(CH₂)ₓ—C₂H₂O, etc., wherein R₄˜R₅ are individually selected from one of the following NH₃ affinity groups: —COOH, —(R)ₓ—COOH wherein R is —(CH₂)ₓ—, —(CH₂)ₓ—COOR wherein R is —(CH₂)ₓ—, —SO₃H—, etc., wherein R₃ and R₆ are individually selected from one of the following groups: H, —CH₃, -Et, -Bu, —COOH, —OH, —NH₂, aniline, sulfonated aniline, OH-aniline, COOH-aniline, etc.

In one preferred example of this embodiment, the R₁˜R₅ groups of the A component and the B component contains at least one CO₂ affinity group and at lest one NH₃ affinity group. The C component is selected from one of the following group: —CH₂—, —CH₂—CH₂—, —(CH₂)ₓ—, —C₆H₆—, —C₆H₄—, —C₁₀H₆—, —C₁₄H₈—, —C₁₈H₁₂—, —CH═CH—, —C═C—, —NH—, —N═, —O—, —CO—, —COO—, —CONH—, —S—, —SO—, —SO₂—, —PO₂—, —P═N—, —BH—, —B(OH)—, etc.

In one preferred example of this embodiment, the mentioned CO₂ reservoir with the functional conducting polymer, and the functional conducting polymer is

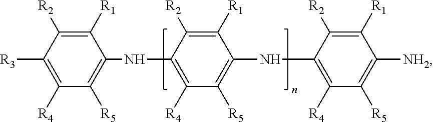

wherein n is an integer equal or more than 1. The method of forming the functional polymer and oligomer according to this embodiment comprises the the following formula (II):

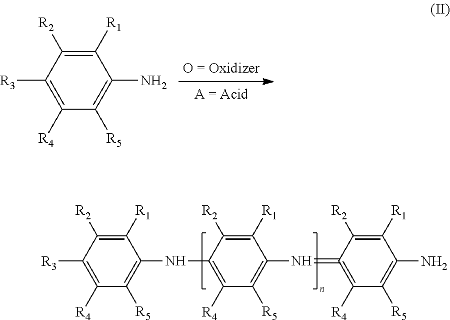

wherein R₁˜R₂ are individually selected from one of the following CO₂ affinity groups: H, —NH₂—, —(R)ₓ—NH₂— wherein R is —(CH₂)ₓ—, —(CH₂)ₓ—CO—NH₂, —B(OH)₂, —(CH₂)ₓ—C₂H₂O, etc., wherein R₄˜R₅ are individually selected from one of the following NH₃ affinity groups: H, —COOH, —(R)ₓ—COOH wherein R is —(CH₂)ₓ—, —(CH₂)ₓ—COOR wherein R is —(CH₂)ₓ—, —SO₃H—, etc., wherein R₃ is selected from one of the following groups: H, —CH₃, -Et, -Bu, —NH₂, aniline, sulfonated aniline, OH-aniline, COOH-aniline, etc.

In the mentioned formula (II), the R₁˜R₅ groups contain at least one CO₂ affinity group and at lest one NH₃ affinity group.

In another preferred example of this embodiment, the functional conducting polymer

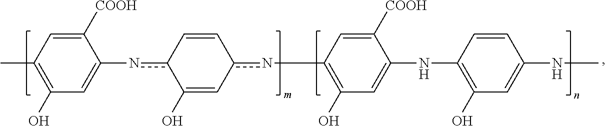

is wherein n is an integer equal or more than 1, and m is an integer equal or more than 1. The formation of the general structure is as the following formula III:

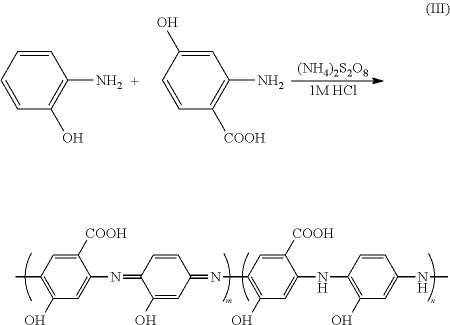

In still another preferred example of this embodiment, the functional conducting polymer

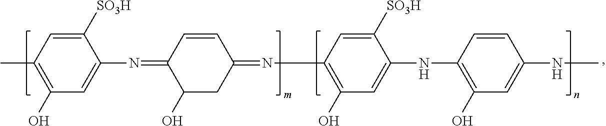

is wherein n is an integer equal or more than 1, and m is an integer equal or more than 1. The formation of the functional conducting polymer is as the following formula IV:

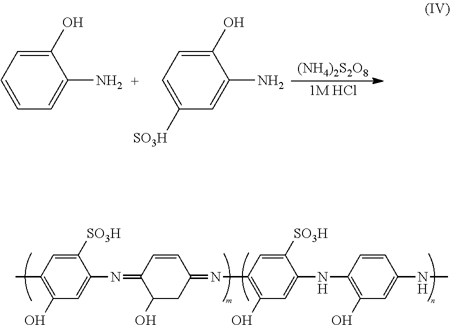

In still another preferred example of this embodiment, the functional conducting polymer is

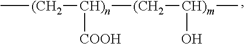

wherein n is an integer equal or more than 1, and m is an integer equal or more than 1. The formation of the chemical structure is as the following formula V:

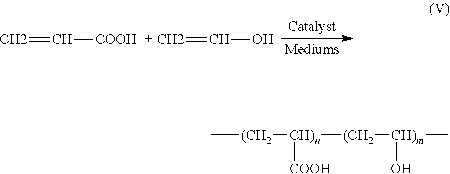

In still another preferred example of this embodiment, the functional conducting polymer is

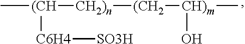

wherein n is an integer equal or more than 1, and m is an integer equal or more than 1. The formation of the general structure is as the following formula VI:

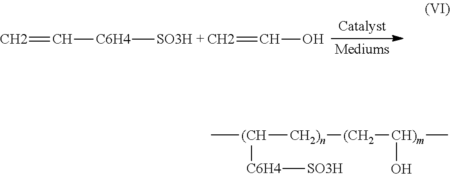

The CO₂ reservoir further comprises an associating compound, and the associating compound can associate with the functional conducting polymer to form a composite, wherein the composite is a functional polymer-associating compound-particle composite.

The associating compound is selected from one of the following types: “p-type” associating compounds supplying “positive charges” to associate the composite, “n-type” associating compounds supplying “negative charges” to associate the composite, protonation associating compounds supplying “protonation” to associate the composite, polymeric associating compounds supplying “polymeric charges” to associate the composite, ionic liquid associating compounds supplying ionic liquid to associate the composite, surfactant associating compounds supplying surfactants to associate the composite, salt associating compounds supplying salt to associate the composite, hydrogen-bonding associating compounds supplying hydrogen bonding to associate the composite.

The particles supply surface and porous areas for the functional conducting polymer and the associating compound, wherein the particles are selected from at least one of the following: clay, silica, alumina, TiO₂, Talc, Boron Nitride (BN), graphite, graphene, carbon nanotubes, carbon nanofibers, active carbons, carbon woods, carbon black, carbon fiber, glass fiber, glass beads, zeolite, polymeric beads, and polymeric particles that supply surface and porous areas for the functional polymer and the associating compound. Typically, these particles include inorganic particles and organic particles in category, and non-reacted and reacted particles in function. In other words, the particle can be selected from one of the following type: inorganic particle acting as media to supply surface and porous areas for the functional polymer and the associating compound, organic particle acting as media to supply surface and porous areas for the functional polymer and the associating compound, non-reacted particle wherein the non-reacted particle will not react with CO₂, and reacted particle wherein the reacted particle will react with CO₂ or generate CO₂.

In one preferred example of this embodiment, the amount of each individual component is variable from the ranges of 0.02 wt. % to 99.95 wt. %, and is added up to a total of 100%. Preferably, the range of the functional polymer is from 20 wt. % to 90 wt. %, the range of the associating compound is from 20 wt. % to 90 wt. %, and the range of the particle is from 5 wt. % to 80 wt. %.

In one preferred example, the composite is preparation by one of the following method or the mix thereof: in-situ polymerization of the functional conducting polymer with the associating compound on the particles, electrically synthesizing the functional conducting polymer with the associating compound on the particles, solution coating the functional conducting polymer with the associate compound on the particles, spray coating the functional conducting polymer with associate the associating compound on the particles, solution blending the functional conducting polymer with the associate compound on the particles, extrusion blending the functional conducting polymer with the associating compound on the particles, vapor phase polymerization of coating the functional associating polymer with the associating compound on the particles.

In another preferred example of this embodiment, the backbone of the functional associating polymer comprises at least a side group —R, wherein R is selected from one of the following: H, —CH₃, —OCH₃, -Et, -Bu, —CH₂—CH₂—NH₂, —(CH₂)ₙ—NH₂, —(CH₂)ₙ—OH, —COOH, —SO₃H—, —B(OH)₂, —OH, as well as a polymeric chains. In this example, the functional associating polymer has a function to chemically associate the CO₂ when R is selected from the amine based chains. The functional associating polymer has a function to physically associate with CO₂ when R is selected from the hydroxyl (—OH) based chains. The functional associating polymer has NH₃ affinity function when R is selected from acidic group (—COOH, —SO₃H—). The functional associating polymer becomes a branched conducting polymer with high CO₂ affiliation groups on both chemical and physical association than the corresponding linear polymer when R is a polymer or conducting polymer.

In one preferred example, the functional polymer-associating compound-particle composite is used as a CO₂ collecting material to remove CO₂ from combustion air and condense CO₂ into dry ice for cooling industry and/or underground CO₂ storage, or react CO₂ with other species to form industrial products and/or fertilizers, wherein the species is selected from the following: CaCO₃, Ca(HCO₃)₂, H₂CO₃, NH₄HCO₃, (NH₄)₂CO₃, KHCO₃, K₂CO₃, etc.

The composite reacts with other miner and volcanic ash materials such as Ca-phosphate (Ca₅(PO₄)₃F), Apatite (Ca₅(PO₄)₃F), Ca-silicate (CaAl₂Si₂O₈), Feldspar Episodes (CaAl₂Si₂O₈), etc. to form fertilizers, kaolinite (Al₂Si₂O₅(OH)₄), Calcite (CaCO₃).

The composite will blend and mix with at least one polymer material to form a blended material, and the polymer material comprises polymers or ceramic precursors comprising one or any combination selected from the group consisting of thermoplastic polymers such as, PS, PVC, PVA, PET, PP, PE, PC, PET, PEN, nylon, PMMA, PAL PEEK, liquid crystal polymer, TPO, PA, PLA, PCL, etc.; thermoset polymers such as, PU, Epoxy, PI, PA, Unsaturated polyester, Vinyl ester, Phenolic, etc.; and ceramic precursors such as silazane lithium salt and titanium tetrachloride (TiCl4), polyureasilazane ceramic precursor, aluminum-containing polycarbosilane, polyaluminocarbosilane, Boron-modied polysilylcarbodi-imides precursors, etc.

The blended material can mix with some blowing agents and co-blowing agents for foaming such as CO₂, N₂, hydrofluorocarbon, fluorocarbon, water, or mixtures thereof. Fluorocarbon and hydrofluorocarbon include CFC11, HCFC 123, HCFC 141b, and commercial products such as Arkema Forane® 134a, R-134a, HFC-134a, DuPont's Dymel® 134a, 152a, etc.

The blended material can form both closed-cell and open-cell structures and bimodal structure when using the particles or the composite, and the blended material can form lighter color foam when using the composite comprising polyaniline, surfactant associating compounds, and dispersed grapheme. The blended material can be a blended resin being used in extrusion foaming and injection molding processes, where water and CO2 will be released to assist the foaming during the extrusion and injection molding.

The composite is used as a gas collecting material such as, SOₓ, NOₓ, H₂S, from the combustion air and condense it into industrial products and/or fertilizers such as NH₄HSO₄, NH₄NO₃, etc.

In addition, the particles or the composite can form nanopapers or nanopapers on a fiber veil or woven and non-woven fabric substrate for coating, filtration and membrane applications.

According to this embodiment, in one preferred example, the mentioned functional polymer-associating compound-particle composite can be blended and mixed with a blending polymer to form a blended material. The blending polymer is selected from polymer material, and/or polymer-ceramic hybrid material.

In one preferred example, the amount of each individual component is varied from 0.02 wt. % to 99.95 wt. %, and is added up to a total of 100%. Preferably, the range of the composite is from 0.05 wt. % to 90 wt. %.

The mentioned functional polymer-associating compound-particle composite can be blended and mixed with the blending polymer by one of the following method: solution blending, melt extrusion, compounding, injection molding, batch foaming, extrusion foaming, or injection molding foaming, etc.

In one example of this embodiment, the mentioned blended material can be mixed with blowing agents and co-blowing agents for foaming. The blowing agents and co-blowing agents are selected from at least one of the following: CO₂, N₂, hydrofluorocarbon, fluorocarbon, water, or mixtures thereof. The mentioned Fluorocarbon and hydrofluorocarbon is selected from the following: CFC11, HCFC 123, HCFC 141b, Arkema Forane® 134a, R-134a, HFC-134a, DuPont's Dymel® 134a, 152a, etc.

In one example of this embodiment, the blended material forms both closed-cell and open-cell structures when the particle of the functional polymer-associating compound-particle composite is selected from the reacted particle. In this example, the amount of the reacted particle is varied from 0.05 wt. % to 10 wt. %.

In another example of this embodiment, the blended material forms lighter color foam.

In another example of this embodiment, the blended material achieves lighter color foam and comprises polyaniline, surfactant associating compounds, and dispersed grapheme. In this example, the amount of each individual component is varied from 0.02 wt. % to 99.95 wt. %, and is added up to a total of 100%.

In one preferred example of this embodiment, the blended material can be used to form a foam presenting a ratio of UV/vis absorption intensity at different wavelengths (450 nm, 500 nm, 610 nm, 660 nm) with the UV/vis absorption intensity for polystyrene (PS at ˜280 nm) less than 0.042 by UV/vis peak ratio method.

In another preferred example of this embodiment, the blended material can be used to form expandable resins through high pressure and/or high temperature water and CO₂ addition processes at a high-pressure chamber. In this example, the blended material and the expandable resins can be used in extrusion foaming and injection molding processes, and water and CO₂ will be released to assist the foaming during the extrusion foaming and injection molding processes.

In still another preferred example of this embodiment, the blended material with reacted particles can be used in extrusion foaming and injection molding processes, and water and CO₂ will be released to assist the foaming during the extrusion foaming and injection molding processes. As an examples, NH₄HCO₃ will release NH₃, CO₂ and H₂O during the extrusion foaming, injection molding.

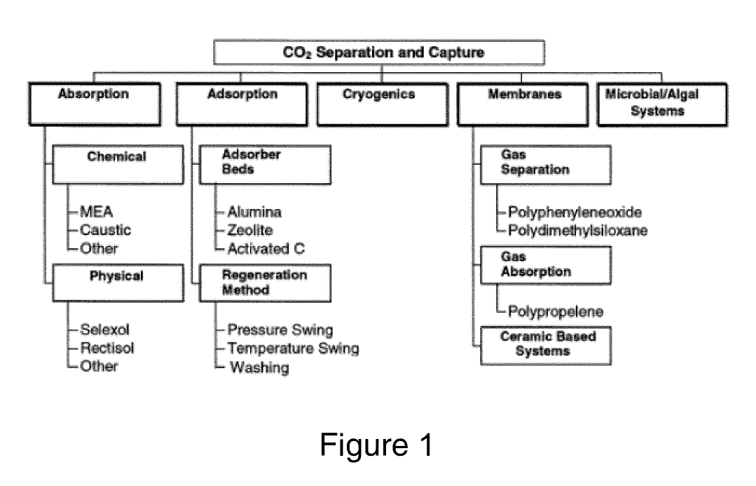

In FIG. 1, it shows that (I) lists the chemical synthesis process of CO₂ functional polymers, wherein A containing at lest one of functional group, which is CO₂ affinity function, and B containing at least one of functional group, which is NH₃ affinity function, (II) lists the chemical synthesis process of functional polyaniline (FPAN), which containing at least one of CO₂ affinity group and at least one of NH₃ affinity group, (III) an example of FPAN, which has a CO₂ affinity group of —OH, and a NH₃ affinity group of —COOH, (IV) an example of FPAN, which has a CO₂ affinity group of —OH, and a NH₃ affinity group of —SO₃H, (V) an example of functional ethylene copolymer, which has a CO₂ affinity group of —OH, and a NH₃ affinity group of —COOH.

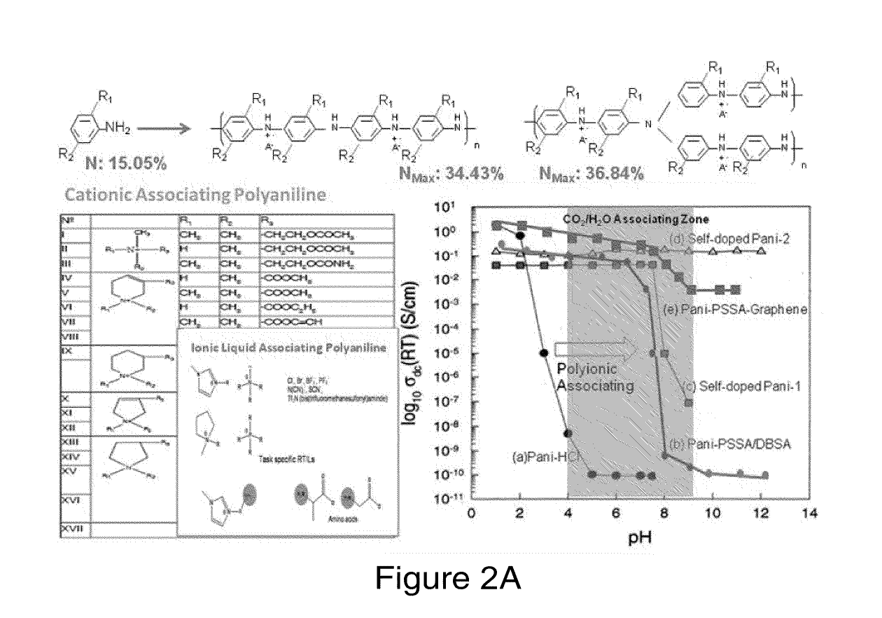

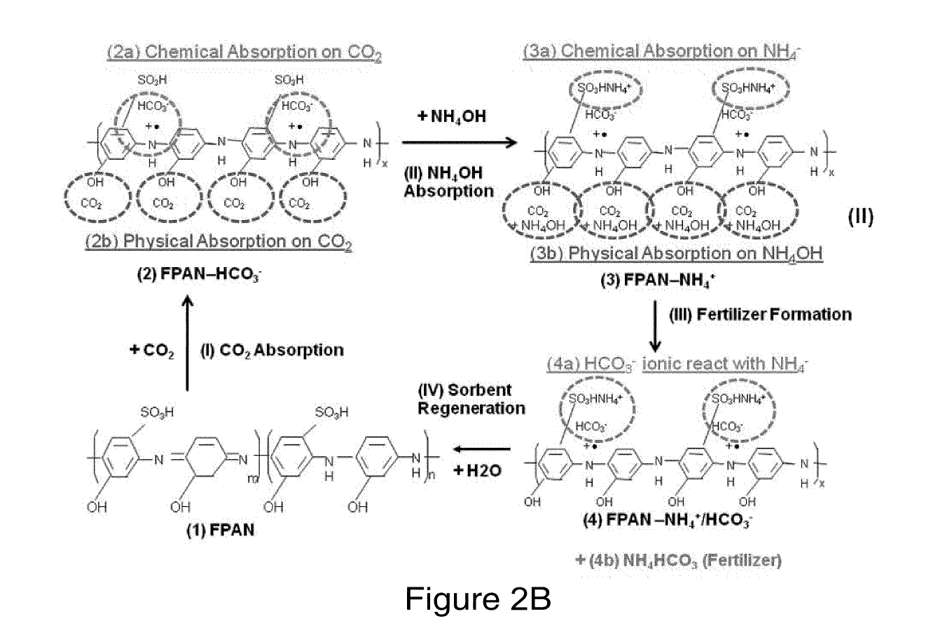

FIG. 2A shows highly branched/side-chain polyaniline their associating compounds (Doping) and pH range control for CO₂ absorption/releasing, wherein in the polyaniline structure, the R would be amine and hydroxyl (—OH) groups, where the amine will act as chemical association to the CO₂, and the hydroxyl group will act as physical association to the CO₂, and FIG. 2B shows the proposed mechanism of CO₂ sequestration and NH₄HCO₃ fertilizer formation.

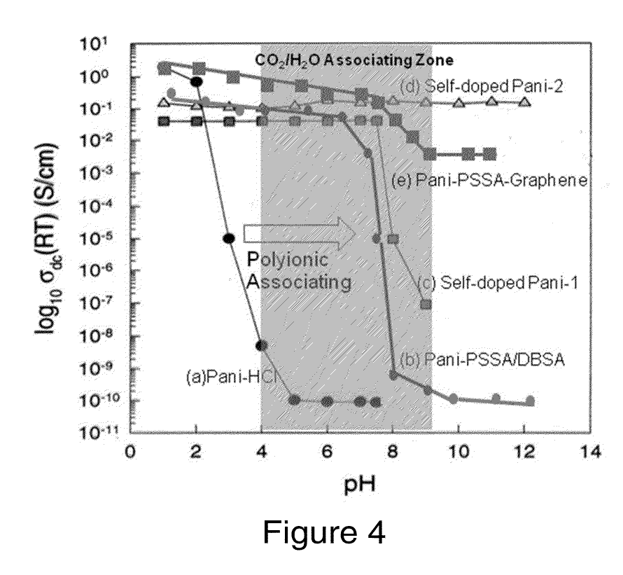

Referred to FIG. 4, the conductivity vs. pH curve from (a) Pani-HCl, (b) Pani-PSSA/DBSA, (c) Self-doped Pani-1, (d) Self-doped pani-2, (e) Pani-PSSA-Graphene.

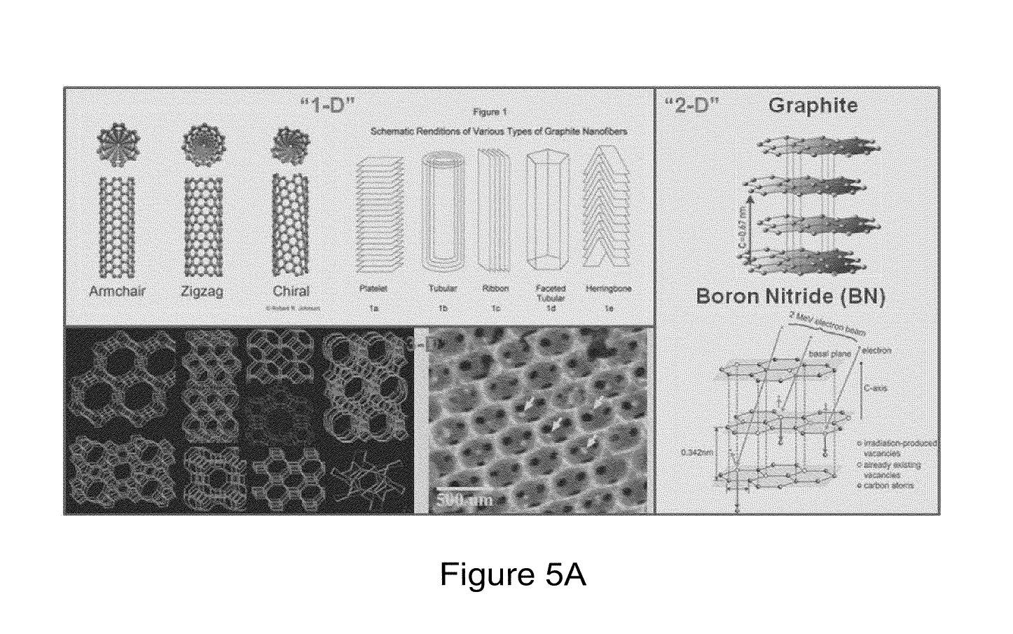

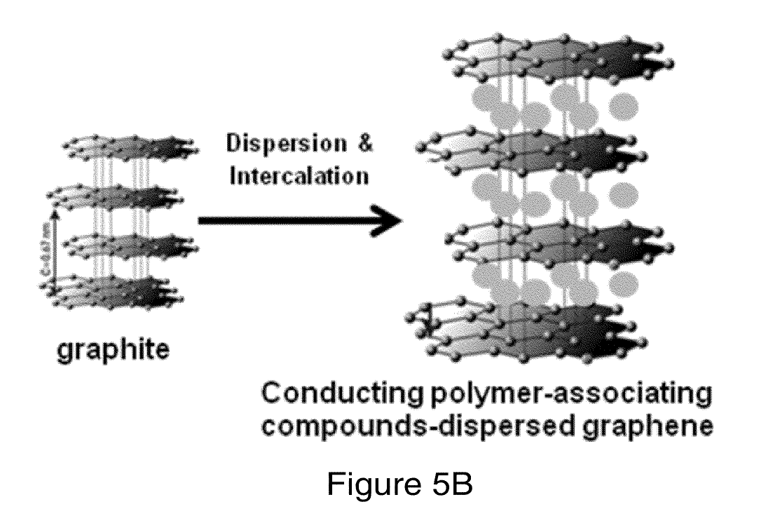

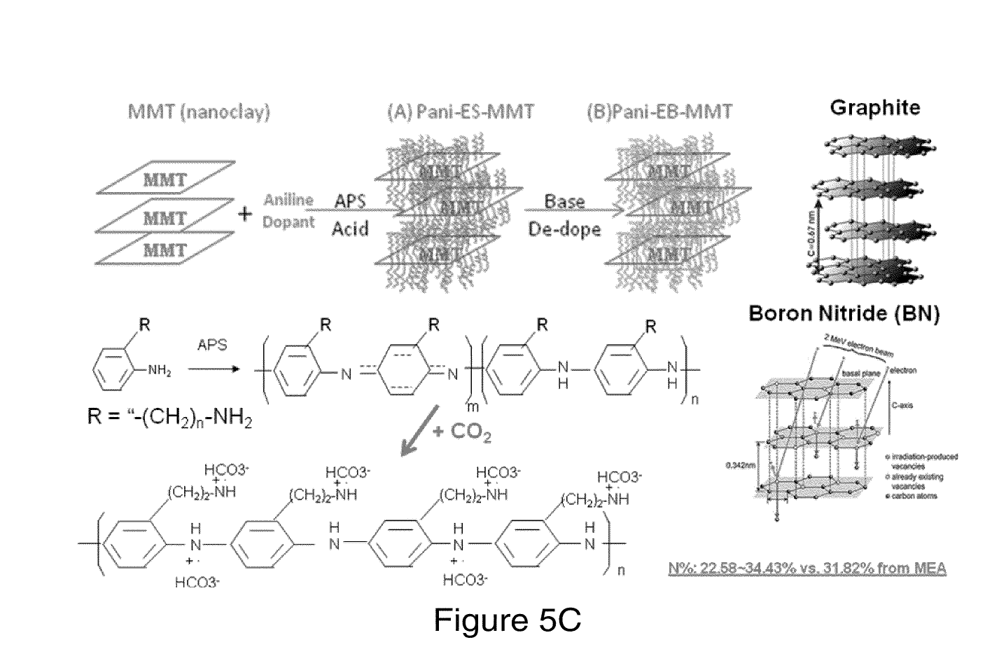

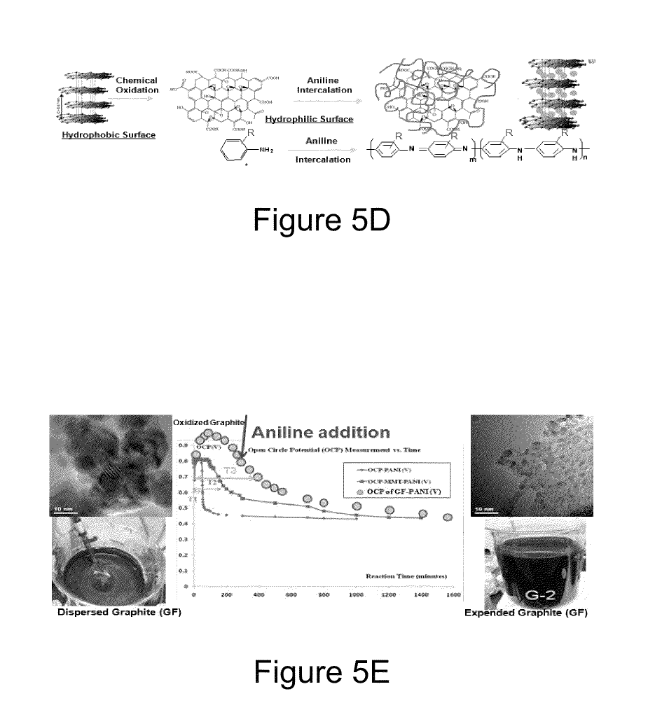

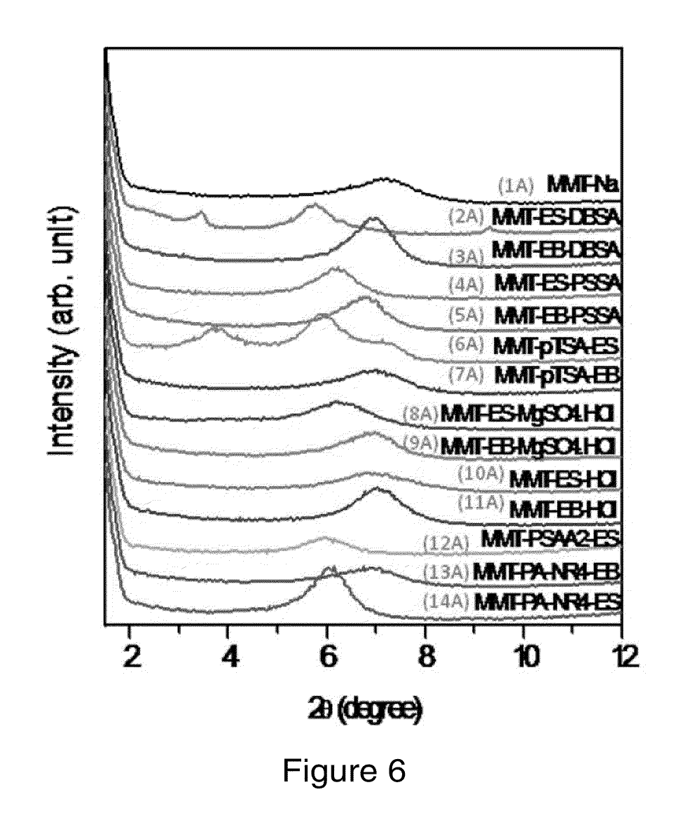

A schematic of the in-situ polymerization of aniline with dispersed grapheme is shown in FIG. 5B. FIG. 5C shows the functional polymer/particle composites and associating with CO₂, in the polyaniline structure, the R would be amine and hydroxyl (—OH) groups, where the amine will act as chemical association to the CO₂, and the hydroxyl group will act as physical association to the CO₂.

FIG. 5D shows the in-situ polymerization of aniline in graphite leading to expended graphite. FIG. 5E shows conducting polymer/particle composites and associating with CO₂. In the polyaniline structure, the R would be amine and hydroxyl (—OH) groups, where the amine acts as chemical association to CO₂, and the hydroxyl group acts as physical association to CO₂.

The experimental results indicated that the EB-MMT-PS blends absorbed more CO₂ and delayed the CO₂ release during foaming comparing to the ES-MMT-PS blends. For Pani-dispersed Graphene-PS blended resin foaming, wherein the experimental results indicated that the resin absorbed more CO₂ (˜8 wt. % vs. ˜6 wt. %) and the final foam shows a lighter color.

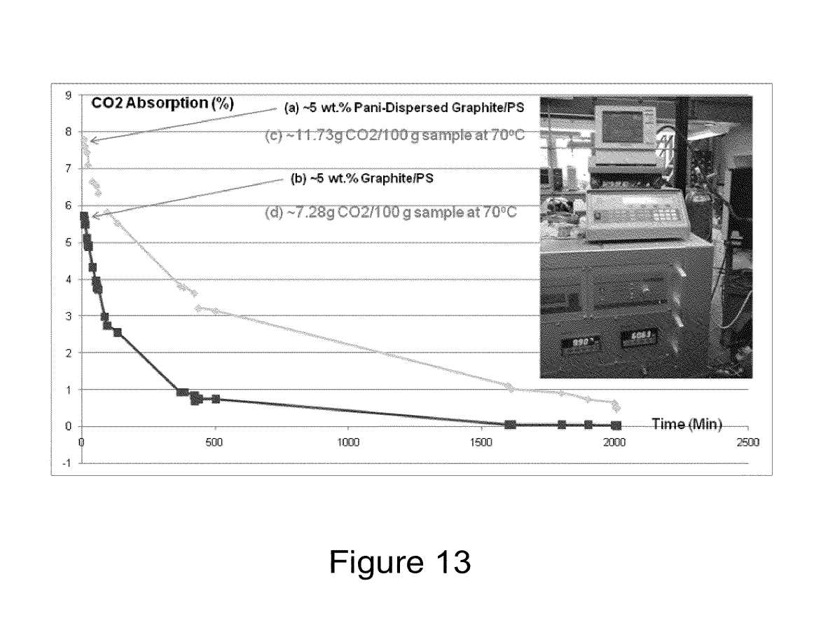

FIG. 13A shows CO₂ absorption test results, wherein (a) presenting 5 wt. % of polyaniline disposed graphite or graphene filled PS blend, and (b) presenting the same amount graphite filled PS blend. The CO₂ foaming condition was at 125° C./2000 psi for 24 hours. It took about 120 seconds to open the pressure chamber. Therefore, there were almost 150 seconds delay to record the first date in the 4-digital balance. There were also some temperature variation during the test. In order to avoid those errors, an accuracy test was conducted at Rubotherm instrument. (c) Sample-A CO₂ absorption was about 11.73 g CO₂/100 g sample at 70° C., and (d) Sample-B CO₂ absorption was about 7.28 CO₂/100 g sample at 70° C.

The CO₂ capture and subsequent conversion into a useful by-product follows a dual chemical loop design where, in loop I, the liquid (or solid) FPAN sorbent absorbs CO₂ in the sequestration reactor to form doped FPAN—HCO₃— at low temperature (<90° C.) and low pressure (<10 psia) with a relatively high speed where the FPAN main chain chemically absorbs the CO₂ and OH— side chains physically absorb CO_{2.} In loop II, the FPAN—HCO₃— absorbs NH₃/H₂O in the fertilizer reactor to form de-doped FPAN—NH₄, at low temperature (<90° C.) and low pressure (<10 psia) with a high reaction rate (in seconds) where the —SO₃H— side chains chemically absorb the NH₃/H₂O and OH— side chains physically absorb NH₃/H₂O. The FPAN—NH₄, quickly (within a few seconds) forms FPAN—NH₄₊/HCO₃₋ (an intermediate ionic pair) to release NH₄HCO₃ fertilizer as a product at low temperature (<60° C.) and low pressure (<10 psia). Since both NH₄₊ and HCO₃₋ stay on the same FPAN polymer chain, therefore, the fertilizer conversion reaction is very fast with a high yield (>95%) because of close molecular level contact of the reactants. The FPAN essentially serve as a catalyst in the doping/de-doping (or charging/discharging) process to allow fertilizer formation under very mild conditions, a unique advantage of the proposed FPAN process. Finally, the FPAN—NH₄₊/HCO₃₋ releases the NH₄HCO₃ fertilizer during cooling and allow the FPAN to be re-used in loop I of the process.

Accordingly, a method of CO₂ capture and conversion is disclosed in the present invention. The method comprises loop I: absorbing CO₂ by the CO₂ reservoir to form doped FPAN—HCO₃₋; and loop II: absorbing NH₃/H₂O by the FPAN—HCO₃₋ to form de-doped FPAN—NH₄₊.

In loop I, the liquid or solid FPAN sorbent absorbs CO₂ in the sequestration reactor to form the doped FPAN—HCO₃₋ at low temperature (<90° C.) and low pressure (<10 psia) with a relatively high speed where the FPAN main chain chemically absorbs the CO₂ and OH— side chains physically absorb CO₂.

In loop II, the doped FPAN—HCO₃₋ absorbs NH₃/H₂O in the fertilizer reactor to form de-doped FPAN—NH₄₊ at low temperature (<90° C.) and low pressure (<10 psia) with a high reaction rate (in seconds) where the —SO₃H— side chains chemically absorb the NH₃/H₂O and OH— side chains physically absorb NH₃/H₂O, and the FPAN—NH₄₊ quickly forms FPAN—NH₄₊/HCO₃₋ to release NH₄HCO₃ fertilizer at low temperature (<60° C.) and low pressure (<10 psia).

### EXAMPLES AND EQUIPMENTS

In the following examples, novel functional conducting polymers and their composite particles were synthesized through different methods in terms of in-site polymerization, vapor phase polymerization, solution blending and coating, spray coating, etc. The polymers and composites were directly used as media for CO₂ absorption through different processes such as absorption towers, fluidized bed reactors, and packed bed reactors. These composite particles can be also blended with polymers through solution blending, thermal blending, thermal extrusion, and plastic compounding, etc. The foaming method can be extrusion foaming, batch foaming or injection molding foaming. The primary blowing agent is CO₂, N₂, hydrofluorocarbon, fluorocarbon, or mixtures thereof. Fluorocarbons and hydrofluorocarbons include CFC11, HCFC 123, HCFC 141b, and commercial products such as Arkema Forane® 134a, R-134a, HFC-134a, DuPont's Dymel® 134a, 152a, etc.

### Experimental Chemicals and Testing Equipments:

Aniline (Ani) from Aldrich was distilled under reduced pressure. Other reagents, such as dopants, hydrochloric acid (HCl), dodecylbenzene sulfonic acid (DBSA), polystyrene sulfonic acid (PSSA), p-Toluene sulfonic acid (pTSA), oxidant (ammonium peroxydisulfate, (APS), and Tetrahydrofuran (THF), were used. PS (Nova 1600) was from NOVA Chemical, Inc.) and the carbon dioxide foaming agent was provided by Praxair. Sodium montmorillonite clay (Na⁺-MMT) was provided by Southern Clay Products Company. The CO₂ absorption level was measured by a Rubotherm instrument and a laboratory setup with 4 digital balances and a thermal control system. The morphology of the foam was observed by scanning electron microscopy (SEM) and photos were obtained on a Phillips XL30 microscope. X-ray diffraction (XRD) patterns were obtained on a Scintag XDS-2000 X-ray diffractometer equipped with CuK X-ray radiation at 45 kV and 20 mA.

### Example-1

### Synthesis of PAN-MMT Nanocomposites

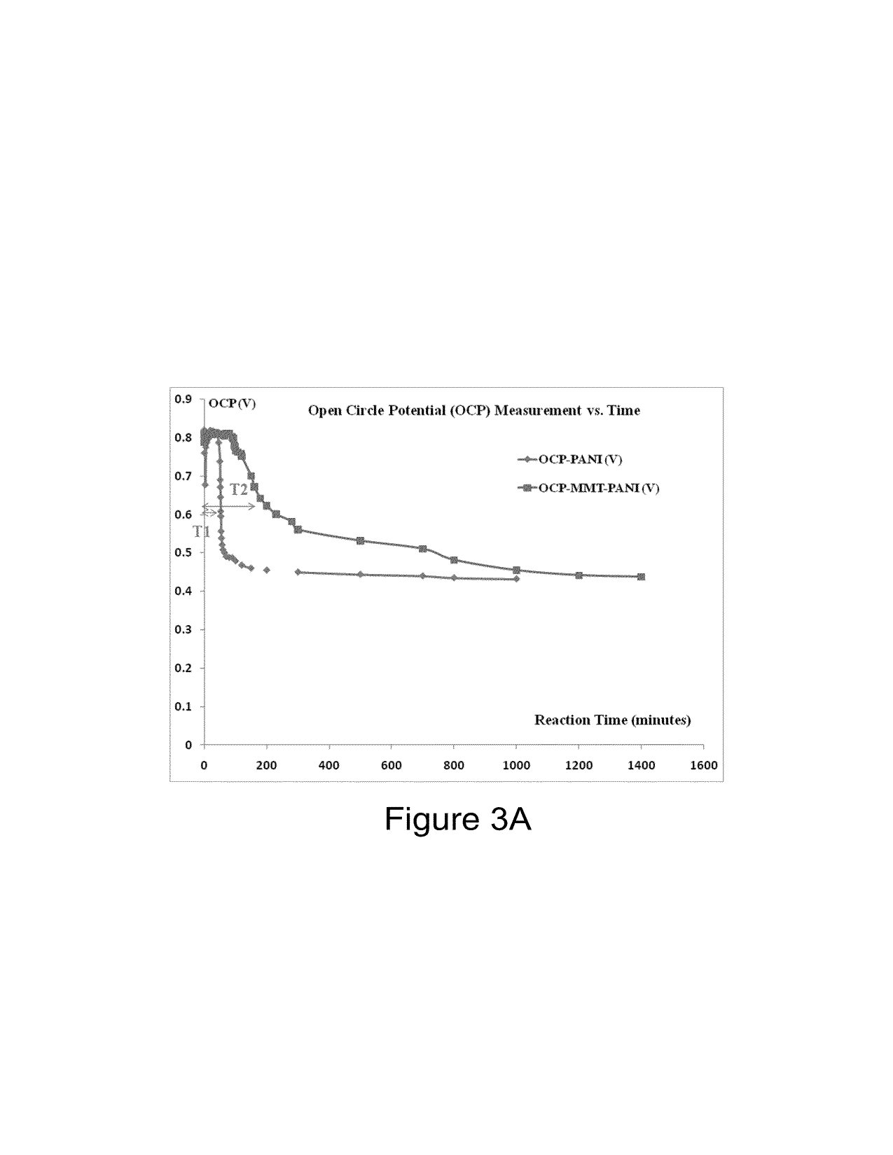

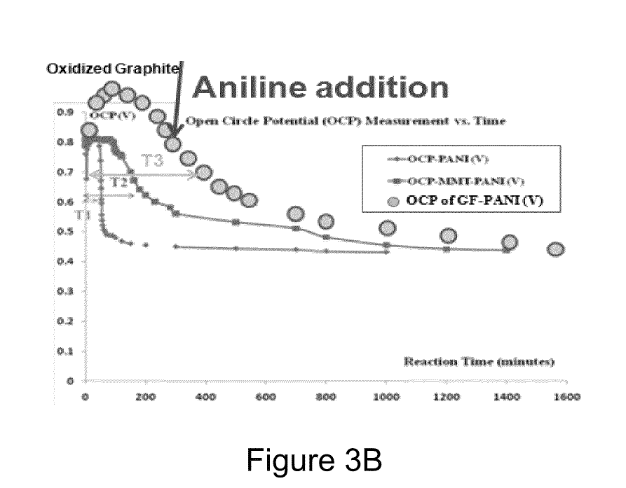

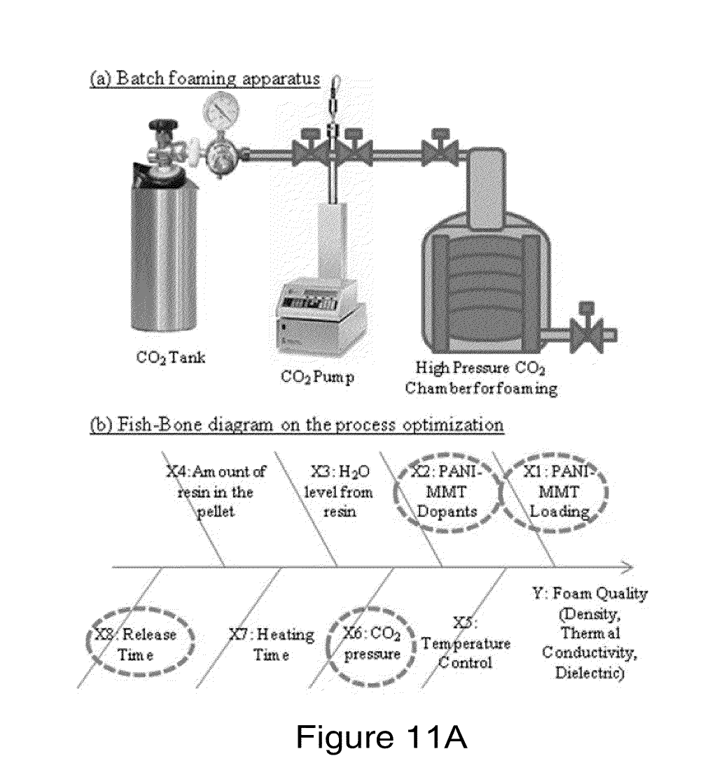

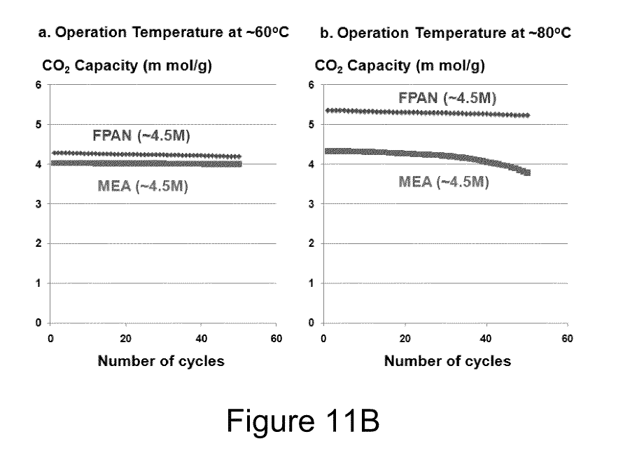

The polyaniline (PAN) modified nanocomposites (MMT) with different dopants were synthesized by self-assembly during the aniline polymerization. For example, the HCl doped PAN-MMT nanocomposite was synthesized as follows: Aniline (6.673 g) was dissolved in a 1000 ml 1M HCl aqueous solution in an ice bath. Then an aqueous solution of APS (13.02 g in 100 ml of deionized water) was added to the above mixture. The polymerization was carried out overnight (˜16 hours) in the ice bath. Green solids of HCl doped PAN-MMT nanocomposites were obtained after rinsing with DI—H₂O three times. Table 1 lists the detailed information on synthesis of PAN-MMT nanocomposites under different acids as associating compounds and open circle potential (OCP) measurements vs. reaction time during the in-situ polymerization were recorded and shown in FIG. 3A. The reaction time (T2) of PAN-MMT was almost three times longer than the reaction time (T1) of pure polyaniline synthesis. A longer reaction time would have the benefit of allowing aniline monomers to disperse deeply into the interlayer of MMT to form a PAN-MMT nanocomposite structure, as evidenced in FIG. 11A from an X-ray Diffraction (XRD) study. The “2-Theta” angle was changed from the original 7.4° (MMT-Na+) to about 3.4° on a MMT-ES-DBSA sample and about 3.8° on a MMT-pTSA-ES sample, indicating that the distance between the MMT layers (d-spacing) has been changed by polyaniline and its associating compound. Additionally, this change was related to the associating compound size and strength, which is important in controlling the MMT dispersion. FIG. 11B shows the thermal stability of FPAN vs. MEA in regard to CO₂ sequestration at 60 and 80° C.

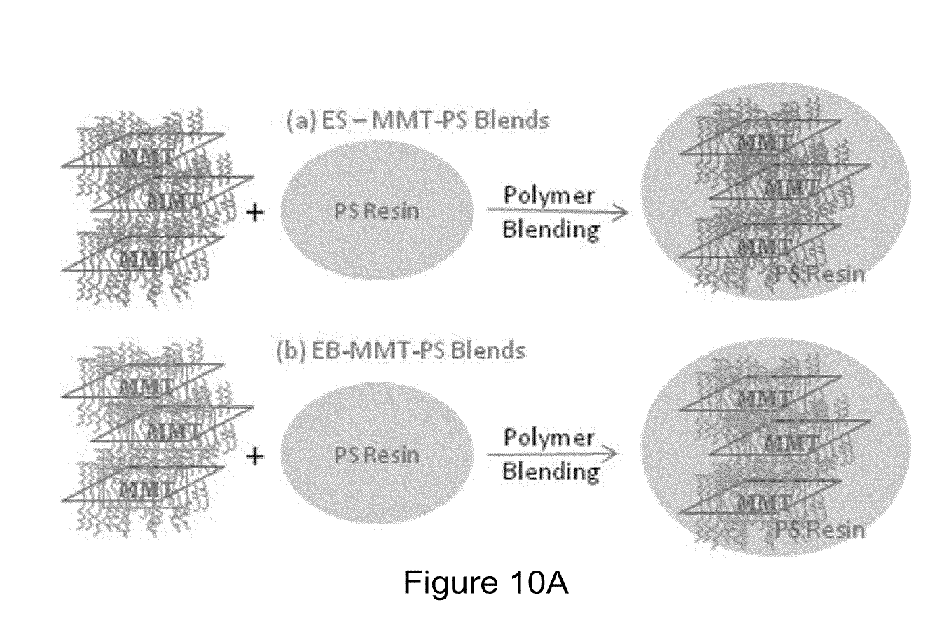

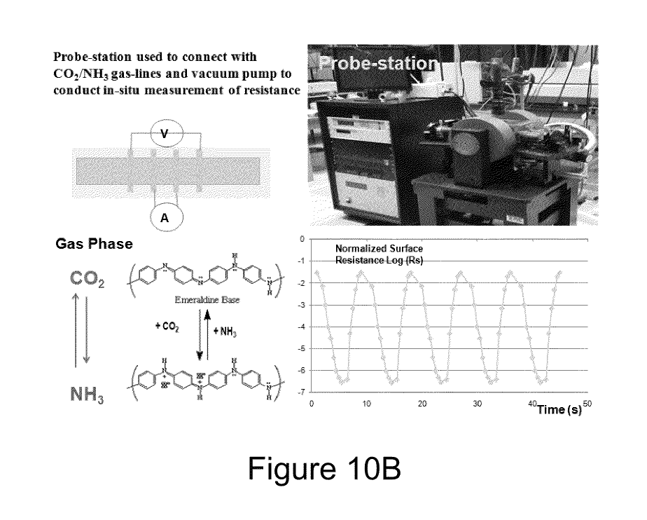

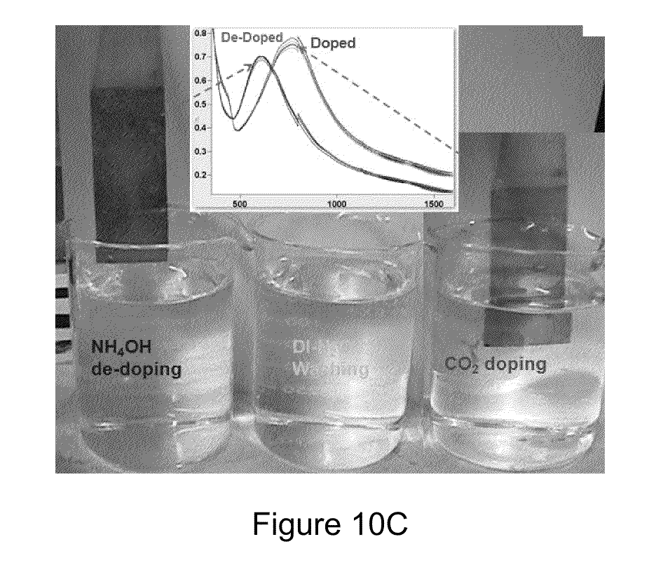

FIG. 10A shows a schematic of the in-situ polymerization of aniline with MMT to form the polyaniline nanoclay composite in doped Emeraldine Salt (ES) form (PAN-ES-MMT) and de-doped Emeraldine Base (EB) form (PAN-EB-MMT). FIG. 10B shows the electric resistance measurements of FPAN—CO₂—NH₃ reaction in gas phase showing very fast reaction rate.

FIG. 10C shows UV/vis spectrum and color changes of FPAN—CO₂—NH₃ reaction in water showing very fast reaction rate (˜10 seconds per cycle), where the “blue color” film is FPAN in-situ polymerized on PET film under the treatment of 0.1 M NH₄OH solution with pH value of 9, and the “green color” film is the same film under the treatment of CO₂/H₂O solution with pH value of 5. This NH₄OH de-doping and CO_{2/}H₂O doping processing was repeated over hundreds times and showing very fast reaction rate (less than 10 seconds per cycle).

FIG. 3B shows the open circle potential (OCP) measurements vs. reaction time during the in-situ polymerization. The “diamond dots” represents the in-situ polymerization of pure aniline (sample PAN—HCl from Table 1 bath-1). The “square dots” represents the in-situ polymerization of aniline with MMT (sample MMT-HCl bath-1 from Table 1 control); The “Origen round dots” (without line) represents the in-situ polymerization of aniline with graphite.

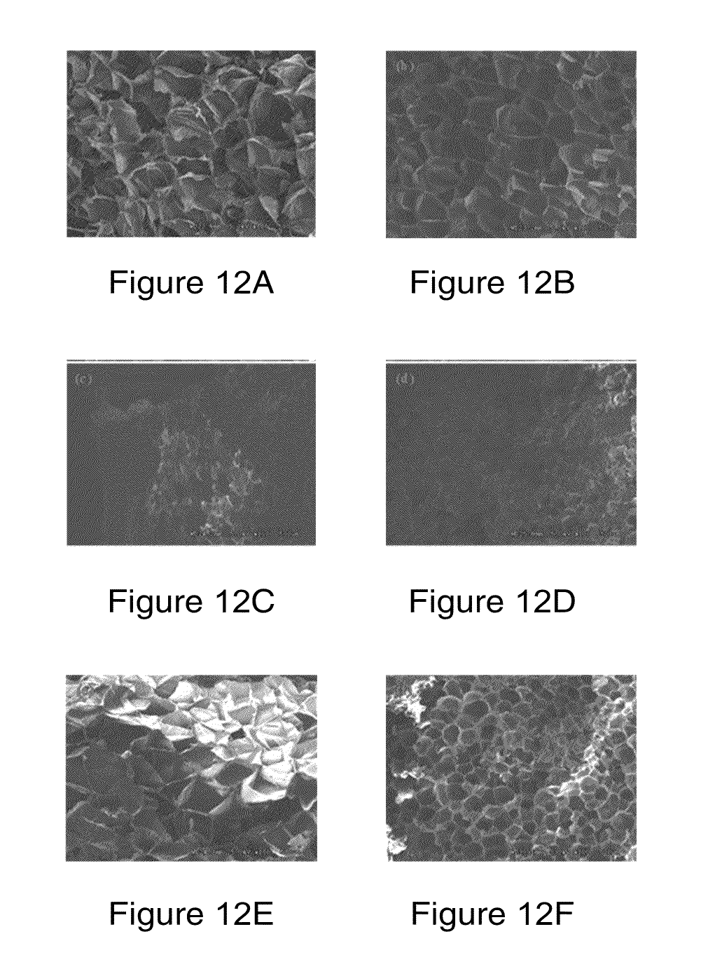

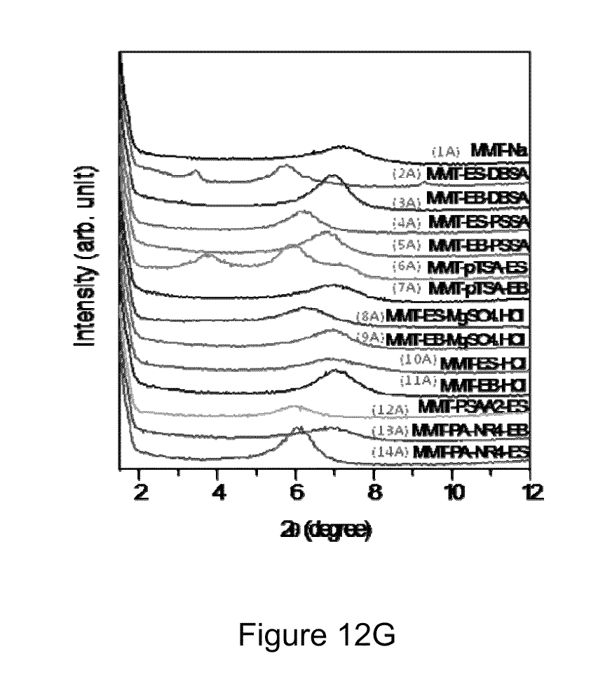

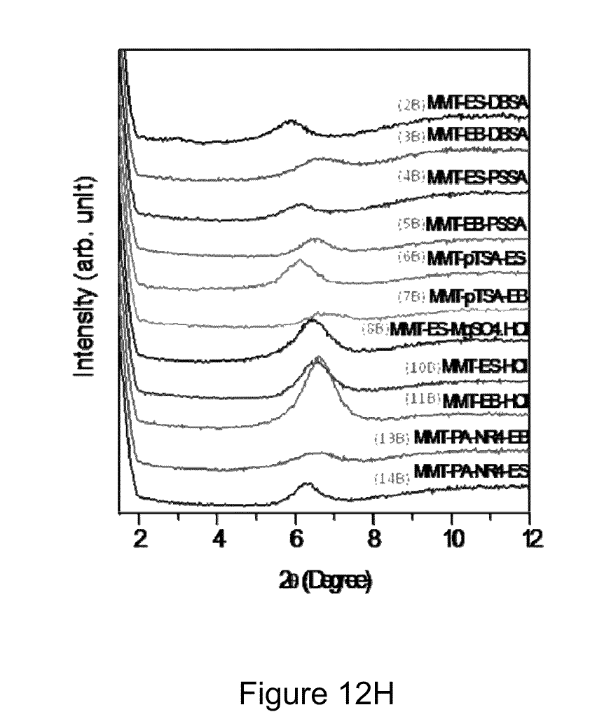

FIGS. 12A to 12F show the SEM micrographs (×100 magnification) of PS filled with (a) DBSA-doped PAN-MMT composites, (b) de-doped DBSA-PAN-MMT composites, (c) PSSA-doped PAN-MMT composites, (d) de-doped PSSA-PAN-MMT composites; (e) PSSA-N+R4 doped PAN-MMT composites, (f) de-doped PSSA-N+R4-PAN-MMT composites.

FIG. 12G shows XRD measurements on

PAN-MMT nanocomposites with various inorganic/organic acids/salts as dopants, where the MMT-Na is the virgin grade of MMT clay from the vendor, MMT-ES-PSSA is PSSA doped PAN-MMT, and MMT-PSSA2-ES is another batch of PSSA doped PAN-MMT composites, where the MMT/Aniline ratio is listed in Table 1.

FIG. 12H shows XRD analysis results on polyaniline nanoclay composites and polystyrene blends with different dopants at both doped form (ES) and de-doped form (EB).

TABLE 1
Data on synthesis of Pani-MMT nanocomposite
particles with different dopants

| Exam- | | Acid | MMT | Aniline | APS | Yield |
|---|---|---|---|---|---|---|
| ple | Samples | category | (g) | (g) | (g) | (%) |
| 1 | Pani-HCl | HCl | 0 | 0.222 | 0.402 | 78.76 |
| | (Control) | | | | | |
| 2 | MMT-HCl | HCl | 1.245 | 0.220 | 0.412 | 86.98 |
| | (bath-1) | | | | | |
| 3 | MMT-HCl | HCl | 3.832 | 6.673 | 13.02 | 87.41 |
| | (bath-2) | | | | | |
| 4 | MMT-pTSA | pTSA | 9.343 | 8.996 | 12.18 | 84.46 |
| 5 | MMT-DBSA | DBSA | 22.94 | 13.83 | 13.98 | 89.70 |
| 6 | MMT-PSSA | PSSA | 6.812 | 6.303 | 9.214 | 88.79 |
| 7 | MMT-PSSA2 | PSSA | 10.087 | 5.716 | 6.982 | 89.16 |
| 8 | MMT-MgSO₄ | HCl— | 9.123 | 10.29 | 13.07 | 85.31 |
| | | MgSO₄ | | | | |

### Example-2

### Graphite Dispersion and DBSA-TBA Intercalation

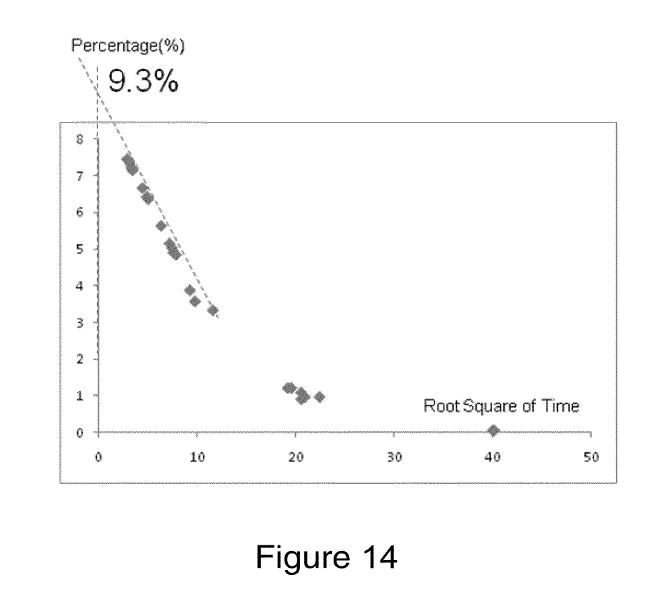

In order to reach a higher degree of intercalation, graphite was dispersed firstly through a combination of physical (sonication), chemical (re-dox reaction) and electro-chemical (electrical re-dox reaction) methods. For example, 25.50 g of graphite was added into 250 ml of fumed H₂SO₄ and then 25.89 g of ammonia persulfate (APS), (NH₄)₂SO₂O₈, and 10.05 g of KMnO₄, to reach a 1.2 V open cycle voltage to ensure graphite dispersion. The reaction medium color was changed from dark black to dark brown, associated with H₂SO₄ fumes. The reaction temperature increased rapidly from room temperature to 100° C. Therefore, an ice batch was used to cool the temperature down to 0-5° C. About 50 g of DBSA was added to the system under high agitation (level-10 magnetic hot plate) for ˜16 hours. Then, the system was filtrated and washed with DI-water three times. The above filter cake was suspended into 1000 ml of DI-water and about 25 g of tetrabutylammonium chloride was added for neutralization. After filtration, drying, and grinding processing, light brown color particles were obtained. The above DBSA-TBA intercalated graphite/graphene particles were not only used as solid and liquid sorbents to conduct CO₂ absorption and NH₄HCO₃ fertilizer conversion test, but also used as CO₂ blowing agent to blend with PS resin for CO₂ foaming. Most importantly, the color of the PS foam is significantly lighter than the color from the same amount graphite as showing from the UV/vis measurement (sample GF1) in FIG. 14 and Table 6.

TABLE 6
UV absorption ratios from UV-vis Spectrum:

| Samples | P450/PS | P500/PS | P610/PS | P660/PS |
|---|---|---|---|---|
| PS-control | 0.041667 | 0.033333 | 0.008333 | 0.016667 |
| OC-Old | 0.058824 | 0.042017 | 0.029412 | 0.02521 |
| OC-New | 0.049587 | 0.049587 | 0.024793 | 0.041322 |
| PaniEB-1% | 0.065574 | 0.04918 | 0.02459 | 0.032787 |
| GR-1% | 0.138211 | 0.073171 | 0.130081 | 0.113821 |
| GR-0.5% | 0.097561 | 0.065041 | 0.073171 | 0.056911 |
| GF1-1% | 0.040984 | 0.036885 | 0.008197 | 0.032787 |
| (sample-2) | | | | |
| GF2-1% | 0.040323 | 0.024194 | 0.008065 | 0.016129 |
| (sample-7) | | | | |
| GF3-1% | 0.041667 | 0.025 | 0.008333 | 0.016667 |
| (sample-4) | | | | |
| GF4-1.5% | 0.036885 | 0.032787 | 0.016393 | 0.028689 |
| (sample-14) | | | | |
| GF4-1% | 0.032787 | 0.02459 | 0.016393 | 0.016393 |
| (sample-14) | | | | |
| MMT-1% | 0.045082 | 0.040984 | 0.02459 | 0.040984 |
| (sample-1) | | | | |
| MMT-0.5% | 0.03252 | 0.020325 | 0.01626 | 0.02439 |
| (sample-1) | | | | |

### Example-3

### Graphite Dispersion and Ionic Liquid Intercalation

Following the above process, 2.59 g of graphite was added to 25 mls of fumed H₂SO₄ and then 2.56 g of ammonia persulfate (APS), (NH₄)₂S₂O₈, to reach a 1.2 V open cycle voltage to ensure graphite dispersion. The reaction medium color changed from dark black to dark brown, associated with H₂SO₄ fumes. The reaction temperature increased rapidly from room temperature to 100° C. Therefore, an ice batch was used to cool the temperature down to 0-5° C. Then, the system was filtrated and washed with DI-water three times. About 6.5 g of butyl methylimidazolium hexafluoro-phosphate ([bmim][PF6]) was added to the system under high agitation (level-10 magnetic hot plate) for ˜16 hours. The above system was washed and filtrated three times with DI-water. The final filter cake was dried in a vacuum oven.

### Example-4

### Synthesis of PAN-Graphene Composites

Using the above method, the polyaniline (PAN) modified graphene composites with different associating compounds were also synthesized by self-assembly during aniline polymerization. In order to reach a high degree of intercalation, graphite was dispersed through a combination of physical (sonication), chemical (re-dox reaction) and electro-chemical (electrical re-dox reaction) methods. For example, 21.25 g of graphite was added into 250 ml of fumed H2SO4 and then 25.67 g of ammonia persulfate (APS), (NH₄)₂S₂O₈, to reach a 1.2 V open cycle voltage to ensure graphite dispersion. The reaction medium color changed from dark black to dark brown, associated with H₂SO₄ fumes. The reaction temperature increased rapidly from room temperature to 100° C. Therefore, an ice batch was used to cool the temperature down to 0-5° C. The polyaniline intercalation reaction was conducted by the following process: aniline (19.6 g) was dissolved in the above mentioned aqueous solution in an ice bath. The polymerization was carried out overnight (˜16 hours) in the ice bath with stirring. Brown-green solids of H₂SO₄ doped PAN-Graphene composites, called “emeraldine salt (ES) composites”, were obtained after rinsing with DI—H₂O three times. A 1 M NH₄OH solution was used to “re-dope” the ES-composite for 3 days. A brown-blue un-doped PAN-graphene composite, “emeradline base (EB) composite”, was obtained after filtration and drying. Besides MMT and graphene, we also successfully intercalated conducting polymer into other inorganic and organic particles, (such as Talc, and Boron nitride (BN) with a similar layer structure as graphite.), TiO₂, BaTiO₃, RuO₂, silica gel micro particles, super branched silica, PS micro beads, PMMA micro beads, PS emulsion, and PVC emulsion, etc. Table 2 lists the detailed information on synthesis of PAN-graphene composites under different acids as associating compounds.

TABLE 2
Data on synthesis of Pani-graphite/graphene
composite particles with different dopants

| Exam- | | Acid | MMT | Aniline | APS | Yield |
|---|---|---|---|---|---|---|
| ple | Samples | category | (g) | (g) | (g) | (%) |
| 9 | Pani-GF-HCl | HCl | 6.08 | 5.59 | 6.78 | 91.26 |
| 10 | Pani-GF- | H₂SO₄ | 21.25 | 19.6 | 25 | 88.93 |
| | H₂SO₄ | | | | | |
| 11 | Pani-GF- | DBSA | 43.25 | 42.53 | 44.58 | 89.16 |
| | DBSA | | | | | |
| 12 | Pani-GF- | pTSA | 6.05 | 5.60 | 6.89 | 87.63 |
| | pTSA | | | | | |
| 13 | Pani-GF-CSA | CSA | 6.10 | 5.67 | 6.92 | 88.17 |
| 14 | Pani-GF-IL* | [bmim] | 6.03 | 5.58 | 6.90 | 85.77 |
| | | [PF₆]. | | | | |
| 15 | Pani-GF- | PSSA | 6.20 | 5.95 | 6.92 | 89.33 |
| | PSSA | | | | | |
| 16 | Pani-GF- | HCl— | 6.33 | 5.83 | 6.98 | 88.42 |
| | MgSO₄ | MgSO₄ | | | | |

The sample 11 from Table 2 PAN-GF-DBSA intercalated graphite/graphene particles were not only used as solid and liquid sorbents to conduct CO₂ absorption and NH₄HCO₃ fertilizer conversion test, but also used as CO2 blowing agent to blend with PS resin for CO₂ foaming. Most importantly, the color of the PS foam is significantly lighter than the color from the same amount graphite as showing from the UV/vis measurement (sample GF3) in FIG. 14, and Table 6.

### Example-5

### Synthesis of PAN-IL-Graphene Composites

It should be mentioned that an ionic liquid (IL) was used as an associating compound during the synthesis as shown in example-14 on Table 2. Room temperature ionic liquids (RTILs) are salts which are liquid around room temperature. The development of these compounds dates back to 1914, with the first preparation of ethylammonium nitrate. More recently, there has been a revival of interest in RTILs due to their potential application as environmentally-friendly and catalytically-active solvents. The main ionic liquid used in this study was butyl methylimidazolium hexafluoro-phosphate ([bmim][PF6]). Hexyl and octyl methyl-imidazolium cations and the tetrafluoroborate anion were also investigated. Many different cations and anions were selected for the study. As an example, 5.65 g of above PAN-graphene composite was treated with 0.51 g of butyl methylimidazolium hexafluoro-phosphate ([bmim][PF6]) to form PAN-IL-graphite composite, where the ionic liquid acted as an associating compound to connect to the polyaniline through ionic interactions.

### Example-6

### Synthesis of PAN-Graphite Composites

Using the above mentioned method, 29.49 g of graphite was added into 2000 ml of 1MHCl and then 34.04 g of ammonia persulfate (APS), (NH₄)₂S₂O₈, to reach a 0.91 V open cycle voltage for graphite dispersion. 29.01 g of aniline was dissolved in the above-mentioned aqueous solution in an ice bath. The polymerization was carried out overnight (˜18 hours) in the ice bath with stirring. Dark-green solids of HCl doped PAN-graphene composites, “emeraldine salt (ES) composites”, were obtained after rinsing with DI—H2O three times. A 1 M NH4OH solution was used to “re-dope” the ES-composite for 3 days. A dark-blue un-doped PAN-graphite composite, “emeradline base (EB) composite”, was obtained after filtration and drying. FIG. 5b shows the results.

### Example-7

### Synthesis of PAN-IL-Graphite Composites

5.03 g of above PAN-graphite composite was treated with 0.49 g of butyl methylimidazolium hexafluoro-phosphate ([bmim][PF6]) to form PAN-IL-graphite composite, where the ionic liquid acted as an associating compound to connect to the polyaniline through ionic interactions.

### Example-8

### PAN—CNF Composite Particles

0.6445 g of CNF particles were added to 120 ml of acetone. A probe sonicator was applied for 30 minutes. In order to control the temperature, an ice bath was used to remove the heat during the sonication. About 12.68 g of aniline was dissolved in 800 ml of 1M HCl. The pre-dispersed CNF/Acetone dispersion was added into the aniline/HCl solution under magnetic agitation. Then, about 14.08 g of ammonia persulfate (APS), (NH₄)₂S₂O₈, was added for polymerization. After 14 hours, “emeraldine salt (ES) —CNF composites” were obtained after rinsing with DI—H₂O three times. A 1 M NH4OH solution was used to “re-dope” the composite for 3 days. A dark-blue un-doped PAN—CNF composite, “emeradline base (EB)-CNF composite”, was obtained after filtration and drying.

### Example-9

### PAN—CNT Composite Particles

0.95 g of CNF particles was added into 100 ml of acetone. A probe sonicator was applied for 30 minutes. In order to control the temperature, an ice bath was used to remove the heat during the sonication. About 9.28 g of aniline was dissolved in 500 ml of 1M HCl. The pre-dispersed CNT/Acetone dispersion was added into the aniline/HCl solution under magnetic agitation. Then, about 11.54 g of ammonia persulfate (APS), (NH₄)₂S₂O₈, was added in for polymerization. After 16 hours, “emeraldine salt (ES) —CNT composites” were obtained after rinsing with DI—H₂O three times. A 1 M NH4OH solution was used to “re-dope” the composite for 3 days. A dark-blue un-doped PAN—CNT composite, “emeradline base (EB)—CNT composite”, was obtained after filtration and drying.

Synthesis of conducting polymer-inorganic/organic particle composites through vapor phase reaction: Example-10: PAN—CaO composite particles: A vapor phase reaction was developed to make composite particles. For example, 9.25 g of CaO particles was added into 250 ml of methanol and then 11.34 g of ammonia persulfate (APS), (NH4)2S2O8, was added in. The mixture was dried and ground into a fine power (particle size less than 200 meshes). 9.3 g of aniline monomer was dissolved in 250 ml 1M HCl methanol (or 1M HCl ethanol, or a mixture) solution. N2 gas was connected to the solution and the vapor phase aniline monomer was connected through the CaO-APS powder. Dark-green PAN—CaO composite particles were made through this aniline vapor phase polymerization.

### Example-11

### PPy-CaO Composite Particles

A polypyrrole (PPy) coated CaO particle was also made through a similar process. For example, 2.50 g of CaO particles were added into 50 ml of methanol and then 3.52 g of FeCl₃ was added. The mixture was dried and ground into fine power (particle size less than 200 meshes). 2.50 g of aniline monomer was dissolved in 50 ml 1M HCl methanol solution. N₂ gas was connected to the solution and the vapor phase aniline monomer was connected through the CaO—FeCl₃ powder. Dark-black PPy-CaO composite particles were made through this pyrrole vapor phase polymerization.

### Example-12

PAN-Hydrogel-CaO Composite Particles

In order to prevent HCl from directly reacting with CaO and improve the ionic and/or water absorption to create a composite medium for CO₂ absorption, crosslinkable hydrogels with ionizable side chains such as 2-hydroxyethylmethacrylate (HEMA), methacrylic acid (MA), tetraethylene glycol dimethacrylate (TEGDMA), and phenypropanolamine (PPA) were chosen as swelling compounds for water and ionic medium absorption agents. For example, 9.45 g of CaO particles were pre-coated with a 1.21 g mixture of crosslinkable hydrogels (MA:HEMA:TEGDMA=1:2:2). The coated CaO particles were then added into 250 ml of methanol and then 11.52 g of ammonia persulfate (APS), (NH₄)₂S₂O₈, was added. The mixture was dried and ground into a fine power (particle size less than 200 meshes). A 9.23 g of aniline monomer was dissolved in 250 ml 1M HCl methanol (or 1M HCl ethanol, or a mixture) solution. N2 gas was connected to the solution and the vapor phase aniline monomer was connected through the CaO-Hydrogel-APS powder. Light-green PAN-hydrogel-CaO composite particles were made through this aniline vapor phase polymerization.

### Example-13

### PAN-Hydrogel-Graphite Composite Particles

In order to create composite particles for high temperature CO₂ absorption, high temperature stable crosslinkable hydrogels with ionizable side chains, such as phenypropanolamine (PPA), were chosen as swelling compounds for water and ionic media absorption agents. For example, 9.52 g of graphite particles was pre-coated with 0.93 g of PPA. The coated graphite particles were then added into 250 ml of methanol and then 11.85 g of ammonia persulfate (APS), (NH₄)₂S₂O₈, was added The mixture was dried and ground into fine power (particle size less than 200 meshes). 9.63 g of aniline monomer was dissolved in 250 ml 1M HCl ethanol solution. N2 gas was connected to the solution and the vapor phase aniline monomer was connected through the graphite-hydrogel-APS powder. Dark-green PAN-hydrogel-graphite composite particles were made through this aniline vapor phase polymerization.

### Example-14

### Synthesis of PAN-Boron Nitride (BN) Nanocomposites

Aniline (9.31 g) was dissolved in a 1000 ml 1M HCl aqueous solution in an ice bath. About 10.55 g of BN particles were added in under high agitation. Then an aqueous solution of APS (11.34 g in 100 ml of deionized water) was added to the above mixture. The polymerization was carried out overnight (˜16 hours) in the ice bath. Light green solids of HCl doped PAN-BN nanocomposites were obtained after rinsing with DI—H₂O three times. The de-doping processing was carried on with 1000 ml of 1 M NH4OH for 48 hours. Then, the filter cake was dried and grinded into powder form.

### Example-15

### Synthesis of Functional Polyaniline (FPAN)

3-OH-aniline (5.12 g) and 2-OH-3-COOH aniline (7.63 g) was dissolved in a 500 ml 1M HCl aqueous solution in an ice bath. Then an aqueous solution of APS (13.77 g in 100 ml of deionized water) was added to the above mixture. The polymerization was carried out overnight (˜24 hours) in the ice bath. Green solids of HCl doped FPAN

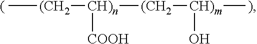

### Example-16

### Synthesis of Functional Polyaniline (FPAN)

3-OH-aniline (10.51 g) and 2-OH-3-sulfonated aniline (15.55 g) was dissolved in a 1000 ml 1M HCl aqueous solution in an ice bath. Then an aqueous solution of APS (23.26 g in 100 ml of deionized water) was added to the above mixture. The polymerization was carried out overnight (˜24 hours) in the ice bath. Green solids are HCl doped FPAN

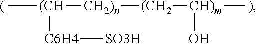

### Example-17

### CO₂ Absorption/Release, NH₄HCO₃ Fertilizer Conversion

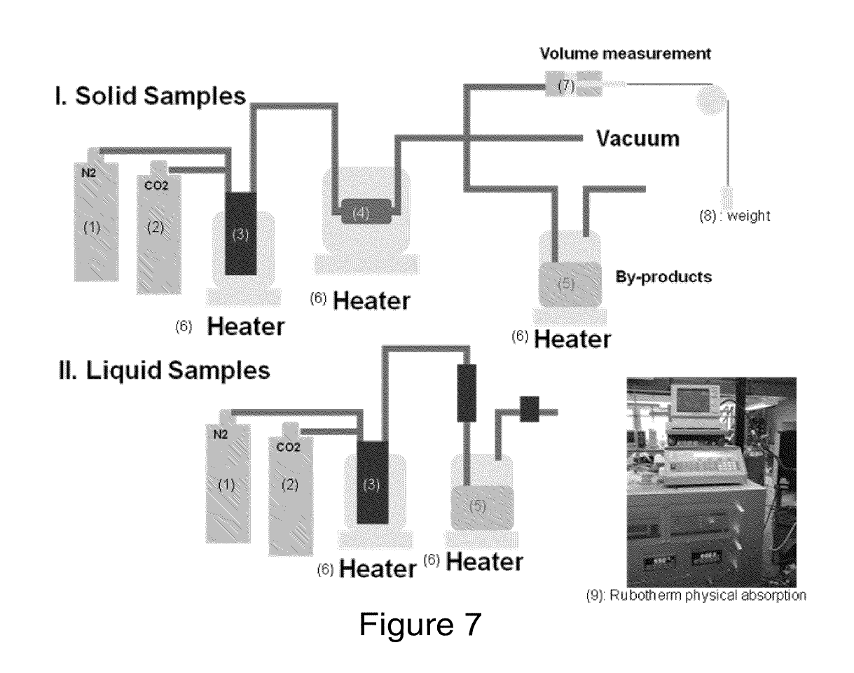

Most existing technologies need to capture CO₂ first and then use the released CO₂ to form industrial products such as polymers, urea fertilizer, etc. For example, the captured CO₂ can react with ammonia (NH₃) to form NH₄HCO₃ through the gas phase reaction. However, it requires high pressure (13-30 MPa) and high temperature (170-200° C.) reaction conditions, implying high energy consumption and high capital equipment investment. Our liquid-solid phase (or liquid-liquid phase) reaction uses a solid phase “polyaniline-HCO₃-composite” (or “polyaniline-HCO₃— composite suspension) and a solution of ammonia hydroxide (NH₄OH) in a reactor. The molecular contact and the catalytic effects provided by FPNA allow one-step and low-energy formation of NH₄HCO₃, a unique feature not achievable by other methods. Lab set-ups were established to test both solid and liquid sorbents on CO₂ sequestration, by-products (i.e. fertilizer) production, and sorbent re-use as shown in FIG. 7. The liquid sorbent test was conducted in a liquid batch where a 15% CO₂/85% N₂ gas mixture was burbling through the solution at 30 ml/min flow rate for 10 minutes. The pre-weighted container was re-weighted after the burbling test to calculate the weight gain from the CO₂ absorption. Then the solution was filtrated through a bush fennel. The filter cake was dried and weight to calculate the product yield. As for the solid sorbent, the pre-weighted solid sample was loaded into a metal tube. Glass wool was used to seal both ends. Then a metal tube was connected to the 15% CO₂/85% N₂ gas mixture at a flow rate of 30 ml/min for 10 minutes. After re-weight the metal tube, we can calculate the amount of CO₂ absorption. The fertilizer was formed on the surface of the solid sorbent. Warm water (˜50° C.) was used to wash off the NH₄HCO₃ and the sample was dried and weighed to calculate the product yield. TAG and Rubotherm absorption methods were used to calibrate the test results. The aforementioned FPAN and their composite particles were used as liquid and solid sorbents to conduct CO₂ absorption and NH4HCO3 fertilizer conversion test as shown in Table 3b. where examples 3,4,5,6,8,9,10,11,12,13, are the corresponding examples 3,4,5,6,8,9,10,11,12,13 from the above. The amino silica (example 1) was used as a control sample for comparison.

TABLE 3b
Data on CO₂ absorption for
Pani-inorganic/organic composites solid particles

| | | CO₂ absorption | CO₂ absorption |
|---|---|---|---|
| | | (wt. %) | (m mol/g) |
| | | on normalized* | on normalized |
| Example | Composite Composition | particles | particles |
| 1 | Amine-silica | 10.74% | 2.44 |
| 2 | Pani-Nanoclay | 13.55% | 3.08 |
| 3 | IL-graphene | 5.68% | 1.29 |
| 4 | Pani-graphene | 13.85% | 3.15 |
| 5 | Pani-IL-graphene | 14.36% | 3.26 |
| 6 | Pani-graphite | 12.22% | 2.78 |
| 7 | Branched Pani-graphite | 15.43% | 3.5 |
| 8 | Pani-CNF | 9.86% | 2.18 |
| 9 | Pani-CNT | 13.81% | 3.13 |
| 10 | Pani-CaO* | 29.72% | 6.75 |
| 11 | PPy-CaO* | 32.51% | 7.39 |
| 12 | Pani-hydrogel-CaO* | 40.89% | 9.29 |
| 13 | Pani-hydrogel-Graphite | 14.61% | 3.32 |
| 14 | Pani-Active Carbon (AC) | 8.87% | 2.02 |
| 15 | Pani-Wood Carbon (Wood) | 13.11% | 2.97 |
| 16 | Pani-IL-Zeolite | 11.72% | 2.65 |

*The test was conducted at TGA from R.T. to 700° C. at 15% CO₂/N₂ condition.
** Normalized particle means using dried particle for the calculation

Table 3c lists the CO₂ absorption using liquid sorbent processing, where polyaniline (PAN), sulfonated polyaniline (SPAN), branched polyaniline (FPAN), and modified polyaniline composite were used for the test. In order to conduct liquid sorbent test, we used MEA as a control sample for comparison.

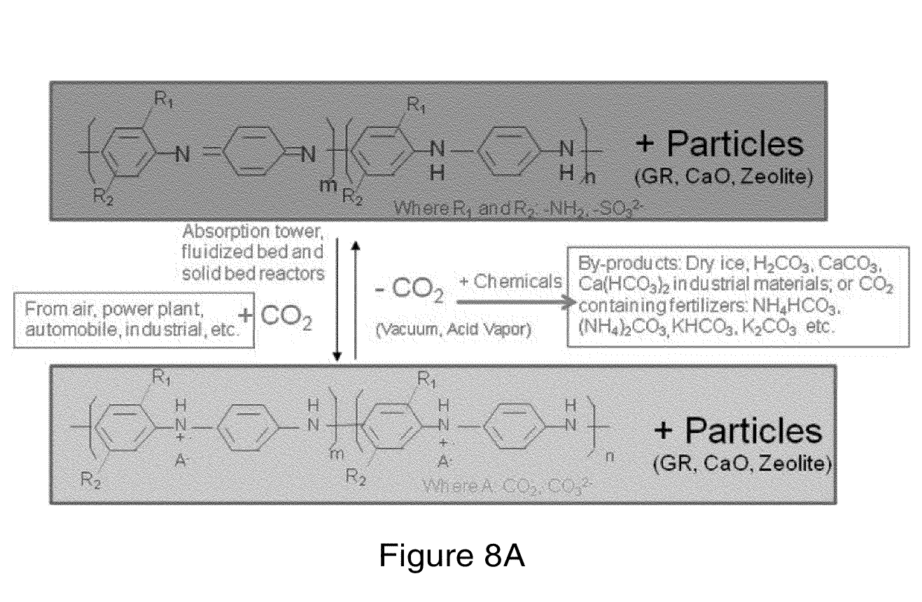

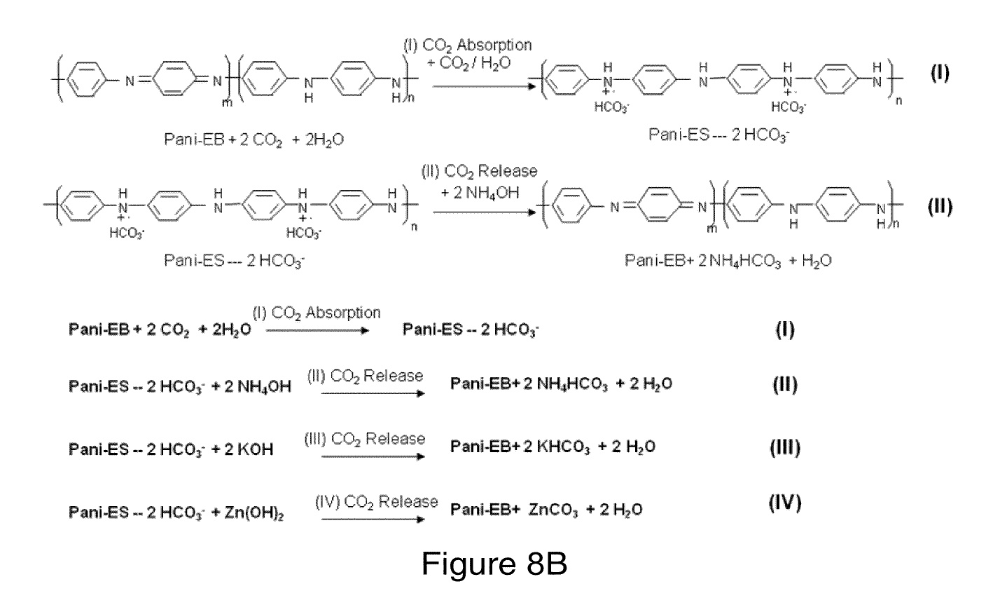

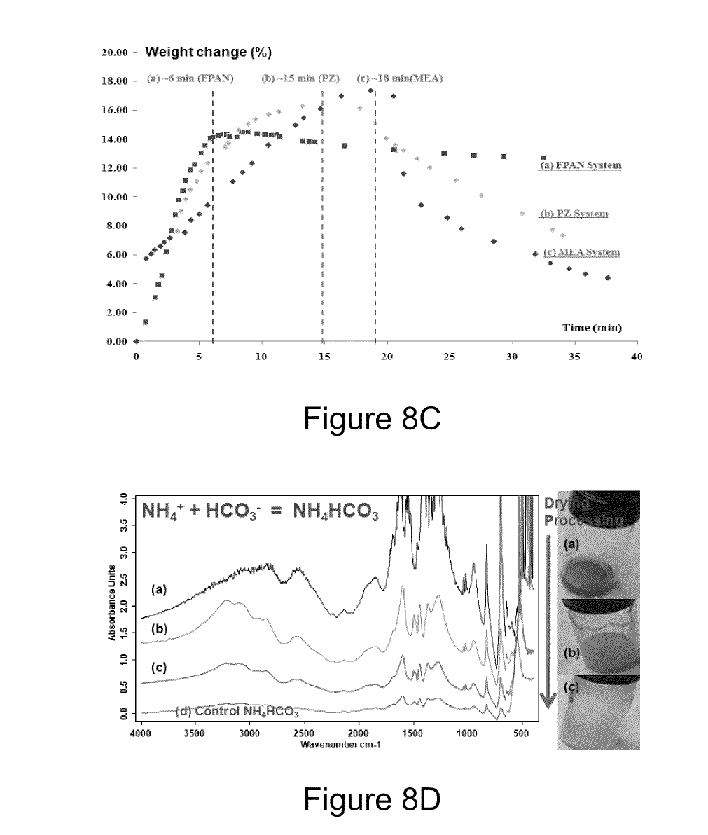

FIG. 8A shows a schematic flow chart of novel composite working hypothesis on CO₂ absorption/release and production of industrial chemicals.

FIG. 8B shows chemical reactions of polyaniline (PAN) with CO₂ absorption and release and conversion to fertilizers, where (i) is PAN-EB/CO₂ absorption reaction, (ii) is PAN-ES/CO₂ release reaction with NH₄OH and NH₄HCO₃ fertilizer is generated as a by-product, (iii) is PAN-ES/CO₂ release reaction with KOH and KHCO₃ fertilizer is generated as a by-product, (iv) PAN-ES/CO₂ release reaction with Zn(OH)₂ and ZnCO₃ fertilizer is generated as a by-product.

FIG. 8C shows the weight changes at three different medium systems at 4M solution concentration, (a) 4M FPAN/water solution, (b) 4M 1-(2-aminoethyl)piperazine (PZ) /water solution, and (c) 4M Mono Ethanol Amine (MEA) /water solution.

The results indicated that the rate of CO₂ absorption is in the order of “FPAN>PZ>MEA”. However, the capacity of CO₂ absorption is in the order of “MEA>PZ>FPAN” if there is enough time (>15 min) for the process. The system viscosity are also different among those three solutions, which are in the order of “FPAN>>Z>MEA” The higher the solution viscosity as well as CO₂ chemical association with FPAN resulted a longer CO₂ release time, therefore, the FPAN solution has a function to “fix” the CO₂ for much longer time comparing to both PZ and MEA solutions. An elevated temperature will speed up the CO₂ absorption and release for all the three solutions. However, both MEA and PZ are high volatile solvents and thermally unstable, which will cause a significant weight loss and efficiency loss after high temperature (>60° C.) and/or long time operation. In the meanwhile, the FPAN has a much higher thermal stability (>220° C.) and a higher chemical stability due to its aromatic polymer backbones. The functional substitution groups will absorpt and release the CO₂ both chemically and physically. In addition, we can use NH₄OH (either NH₃ gas, or NH₄OH liquid) to chemically “de-dope” the CO₂ doped FPAN to form NH₄HCO₃ as a fertilizer. The “de-doped” FPAN will be re-used for another cycle of CO₂ absorption and release. This process is not limited to NH₄OH. Pretty much all of the base type materials (inorganic and organic) can have the functional to “de-dope” the FAPN and formed corresponding by-products. As mentioned, in the above, the “de-doped” FPAN could be reused for many cycles. Certainly, we also can follow MEA's processing to release the CO₂ from doped-FPAN physically by heating and/or vacuum processing. By a certain design in the processing, we can fully utilize the FPAN higher viscosity characters to a benefit. As we know, the higher the viscosity will be benefit for CO₂ absorption to avoid the CO₂ loss during the process; then, we could use elevated temperature and vacuum system to release the CO₂ for concentricity. In addition, we also can modify the polymer chain length and side-chain branching to adjust the FPAN solution viscosity, CO₂ affinity, chemical and thermal stabilities.

FIG. 8D shows the FTIR studies of concentrated solution after NH₄OH de-doping process, where (a) is a concentrated NH₄HCO₃ solution, (b) more concentrated (wet-crystal) NH₄HCO₃ solution and (c) dried NH₄HCO₃ powder. The FTIR spectrum of final dried NH₄HCO₃ powder is matched with the (d) control NH₄HCO₃ ordered from Aldrich.

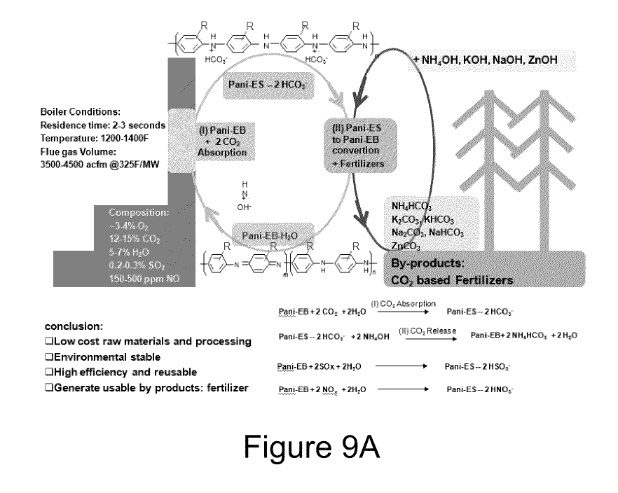

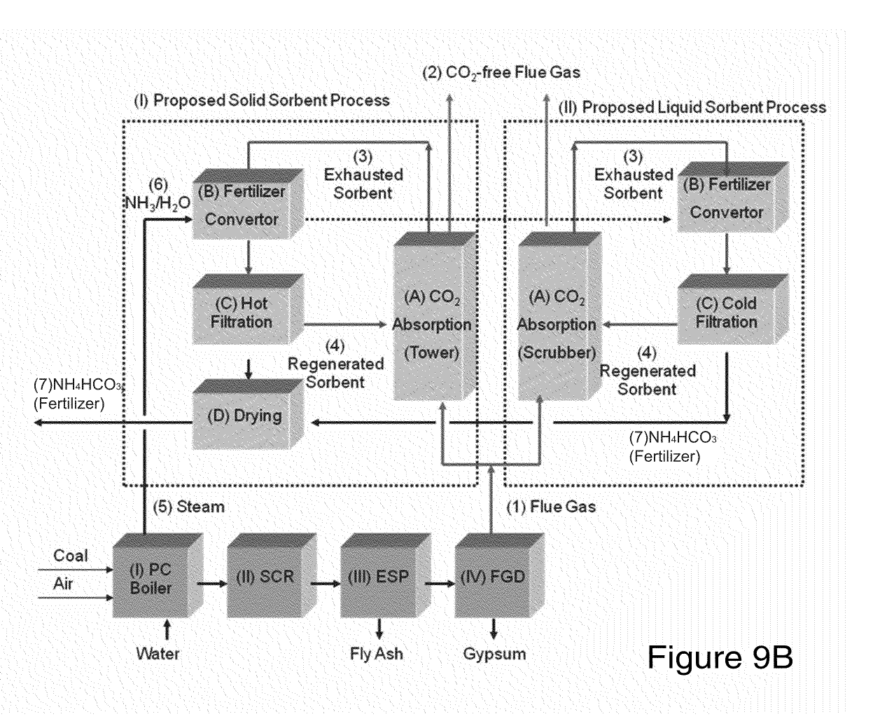

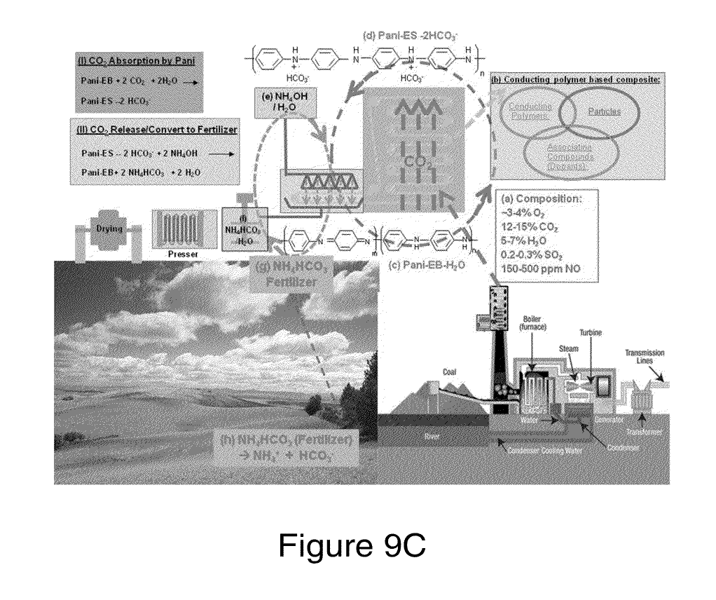

FIG. 9A shows a schematic illustration of CO₂ sequestration and conversion to fertilizers.

FIG. 9B show a schematic process diagram of CO₂ sequestration and conversion to fertilizer, the left process (I) for the solid sorbent and the right process (II) for the liquid sorbent.

FIG. 9C shows a schematic illustration of a continuous belt design of CO₂ sequestration process, where the CO₂ active material is coated on a substrate (glass mat as a example) and the substrate moves like a transition belt to absorb the CO₂ from any CO₂ rich area, and release the CO₂ through NH₄OH washing. The washed solution is then dried to form fertilizer, and the regenerated substrate can be reused for next cycle.

TABLE 3C
Pani-inorganic/organic composites liquid
samples:

| | | CO₂ | CO₂ |
|---|---|---|---|
| | | absorption | absorption |
| | | (wt. %) | (m mol/g) |
| | | on | on |
| | | normalized* | normalized |
| Example | Composite Composition | particles | particles |
| 1 | Ethanolamine (MEA) | 17.74% | 4.03 |
| 2 | NH₄OH | 13.55% | 3.08 |
| 3 | Polyethylenimine (PEI) | 15.68% | 3.56 |
| 4 | Sulfonated polyaniline (SPAN) | 15.88% | 3.61 |
| 5 | polystyrene sulfonate-NH₄⁺ | 14.36% | 3.26 |
| 6 | Polyethyleneamine (PEA) | 13.46% | 3.06 |
| 7 | Polyacrylamide (PAA) | 12.23% | 2.78 |
| 8 | Tetrafluoroethylene | 13.86% | 3.15 |
| | (Nafion)-NH₄⁺ | | |
| 9 | Hydrophilic polyacrylamide | 17.81% | 4.05 |
| | gel (Aquamid) | | |
| 10 | PolyAMPS-NH₄⁺ | 15.72% | 3.57 |
| 11 | Branched Pani-PSSA-NH₄⁺ | 20.51% | 4.66 |
| 12 | Pani-polystyrene sulfonate-NH₄⁺ | 18.89% | 4.29 |
| 13 | SPAN-Polyethylene amine | 16.73% | 3.79 |
| | (PEA) | | |
| 14 | SPAN-Polyacrylamide (PAA) | 15.61% | 3.55 |
| 15 | Pani-Tetrafluoroethylene | 15.87% | 4.29 |
| | (Nafion)-NH₄⁺ | | |
| 16 | SPAN-Hydrophilic | 18.11% | 4.12 |
| | polyacrylamide (Aquamid) | | |
| 17 | SPAN-PolyAMPS-NH₄⁺ | 17.72% | 4.03 |

*Normalized particle means using dried particle for the calculation

Table 3d presents some results on NH₄HCO₃ fertilizer conversion for both liquid and solid sorbents. High conversion yields were obtained with high product purity as listed in Table 3f by elemental analysis. FIGS. 8A and 8B show all chemical reactions for CO₂ sequestration and fertilizer conversion.

TABLE 3d
Conversion of captured CO₂ to NH₄HCO₃ for
liquid and solid sorbents

| | | CO₂ | NH₄HCO₃ | |
|---|---|---|---|---|
| | Composite | absorption | Production | Conversion |
| | Composition | (g) | (g) | yield (%) |
| I. Liquid | | | | |
| Example | | | | |
| 1 | FPAN | 34.05 | 32.39 | 92.3 |
| | (15 wt. % solution) | | | |
| 2 | Pani-PSSA-NH₄⁺ | 33.89 | 32.37 | 95.5 |
| II. Solid | | | | |
| Example | | | | |
| 3 | FPAN-Nanoclay | 24.33 | 20.76 | 85.3 |
| 4 | FPAN-graphene | 27.65 | 24.53 | 88.7 |
| 5 | Pani-graphene | 24.89 | 21.55 | 86.6 |
| 6 | Pani-CNT | 24.73 | 22.87 | 92.5 |
| 7 | Pani-Activated | 23.46 | 20.74 | 88.4 |
| | Carbon | | | |
| | (Wood Carbon) | | | |

* Normalized particle means using dried particle for the calculation

TABLE 3f
Elemental analysis results of CO₂
regenerated NH₄HCO₃:

| | C % (purity) | H % (purity) | N % (purity) | |
|---|---|---|---|---|
| | Measured/ | Measured/ | Measured/ | Total |
| Samples | Calculated | Calculated | Calculated | (purity) |
| Standard | 15.15/15.19 | 6.41/6.37 | 17.45/17.72 | 39.01/39.28 |
| NH4HCO3* | | | | (99.31%) |
| From liquid | 15.01 | 6.35 | 17.25 | 38.61 |
| process | (98.82%) | (99.69%) | (97.34%) | (98.29%) |
| From solid | 15.12 | 6.31 | 17.62 | 39.04 |
| process | (99.54%) | (98.90%) | (99.44%) | (99.39%) |

*Purchased from Aldrich

FIG. 10A shows the very fast CO₂ sequestration and reaction with NH3 measured by the electric resistance analysis. The conducing nature of PAN and FPAN allows them to serve as a sensor in the CO₂ sequestration and fertilizer conversion process. FIG. 11A shows that FPAN is much more thermally stable than MEA based on the CO₂ sequestration capacity at 60 and 80° C. through many cycles.

### Example-18

### Preparation of PAN-MMT-PS Blends

The preparation of CO₂ expandable PAN-MMT-PS blends (or beads) was conducted through a polymer solution blending process. About 5 wt. % of polyaniline modified MMT solid (both PAN-ES-MMT and PAN-EB-MMT) was pre-dispersed in a tetrahydrofuran (THF) solution under magnetic stirring and followed by sonication for about 30 minutes. Then about 95 wt. % of PS resin was added in the pre-dispersed solution following the same process. The polymer solution/blend was dried in a hood at room temperature while being magnetically stirred overnight (˜16 hours). Then the nanocomposites were further dried in a vacuum oven at 180° C. for 12 hours to form blended PAN-MMT-PS resin (or beads). It should be noted that the dispersibility of doped PAN-MMT, Emeraldine Salt (ES) form of polyaniline, was not as uniform as the dispersibility of de-doped PAN-MMT, Emeraldine Base (EB) form of polyaniline. This is because the doped form of PAN had a relative lower solubility than the un-doped form. However, the doped form of PAN is conductive, which could be significant in some industrial applications. Polymer nanocomposites were processed into a rod-like shape with a diameter of 2 mm and a length of 15 mm by a micro-compounder (DACA Instruments). Then, the sample was placed in a round sharp steel frame to form a size of 30 mm in diameter and 3 mm in thickness. PAN-MMT/PS blended pallets were prepared through thermal press processing at ˜180° C. under a pressure of ˜200 psi for the XRD study and batch foaming processing. FIG. 10A shows XRD analysis results on polyaniline nanoclay composites and polystyrene blends with different dopants at both doped form (ES) and de-doped form (EB). In order to understand the dispersion mechanism, Table 4 summarizes the “2-Theta” angle from XRD from both polyaniline nanoclay composites and polyaniline nanoclay-polystyrene blends. It was found that (1) polyaniline in-situ polymerization with nanoclay did not result in “fully exfoliated” nanoclays as expected initially. This is because the synthesis of polyaniline requires an acidic medium condition, i.e., pH<3, while the optimized nanoclay dispersion condition is neutral, i.e., 6<pH<8; (2) the “2-Theta” angle, which relates to the d-spacing between the nanoclay layers, from XRD shows a broad range from 7.4° to 3.4° by various dopants; (3) the doped polyaniline (ES form) has a lower “2-Theta” angle comparing to its de-doped form (EB form) m) in both nanocomposites and PS blended forms. This is because the dopants and polyaniline act as a “spacer” to fill in the interlayer space between the nanoclay layers. However, the de-doping process chemically removes some dopants away from the nanocomposites, resulting in some loss of the “spacer”, therefore, reducing the interlayer distance between the nanoclay layers; (4) the “2-Theta” angle was reduced in most cases after blending with polystyrene. This is because some polystyrene polymer chains had interpenetrated into the layers of polyaniline nanoclay composites, therefore, enlarging the d-spacing between the nanoclay layers.

TABLE 4
Summary of XRD for Pani-MMT nanocomposites and Pani-MMT-PS blended polymers:

| Experimental | | | | | | |
|---|---|---|---|---|---|---|
| Numbers | Acid category | Doping | Pani-MMT | 2 Theta | PS Blend | 2 Theta |
| 1 | MMT-Na(virgin) | | 1A | 7.4 | | |
| 2 | DBSA-Acid | ES* | 2A | 3.4; 5.8 | 2B | 5.8 |
| 3 | DBSA-NH₄ salt | EB | 3A | 7.2 | 3B | 6.8 |
| 4 | PSSA-Acid | ES | 4A | 6.2 | 4B | 6.2 |
| 5 | PSSA-NH₄ salt | EB | 5A | 6.8 | 5B | 6.8 |
| 6 | pTSA-Acid | ES | 6A | 3.8; 5.9; 7.4 | 6B | 6.2 |
| 7 | pTSA-NH₄ salt | EB | 7A | 7.2 | 7B | 6.8 |
| 8 | NH4—MgSO₄ salt | EB | 8A | 7.2 | 8B | 6.7 |
| 9 | HCl—MgSO₄ acid | ES | 9A | 6.2 | | |
| 10 | HCl- Acid | ES | 10A | 7.0 | 10B | 6.7 |
| 11 | NH₄ Cl salt | ES | 11A | 7.2 | 11B | 6.8 |
| 12 | PSSA-acid | ES | 12A | 6.0 | | |
| 13 | NR₄Cl—HCl acid | ES | 13A | 6.0 | 13B | 6.8 |
| 14 | NR₄Cl—NH₄ salt | EB | 14A | 7.0 | 14B | 6.2 |

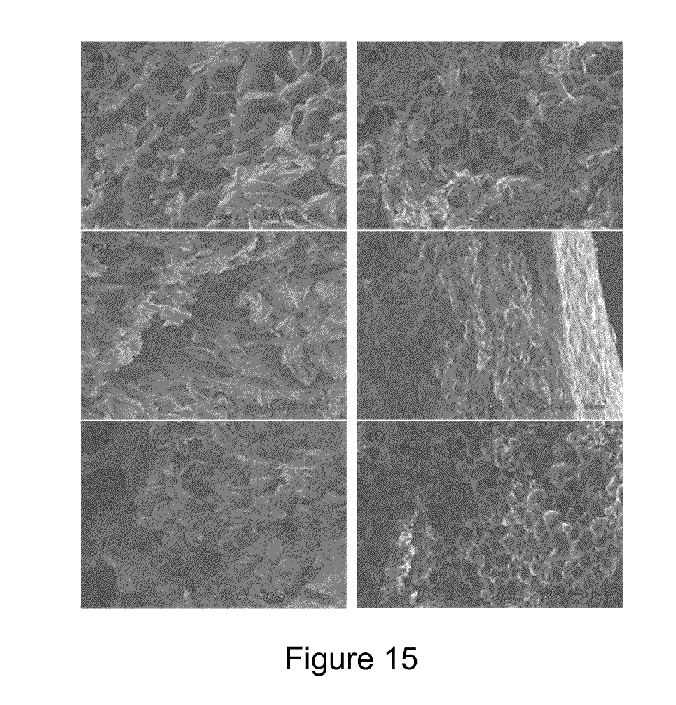

FIG. 15 shows SEM micrographs (×100 magnification) of PS filled with (a) HCl-doped PAN-MMT composites, (b) de-doped HCl-PAN-MMT composites, (c) pTSA-doped PAN-MMT composites, (d) de-doped pTSA-PAN-MMT composites; (e) MgSO4-HCl doped PAN-MMT composites, (f) de-doped MgSO4-PAN-MMT composites. The left 3 SEM pictures show an open-cell structure, and the right 3 SEM pictures show a closed-cell structure.

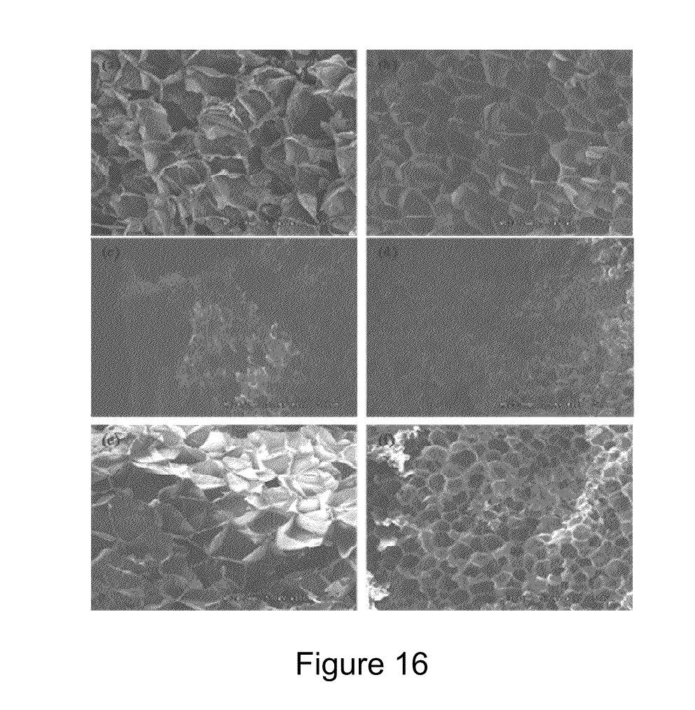

FIG. 16 shows SEM micrographs (×100 magnification) of PS filled with (a) DBSA-doped PAN-MMT composites, (b) de-doped DBSA-PAN-MMT composites, (c) PSSA-doped PAN-MMT composites, (d) de-doped PSSA-PAN-MMT composites; (e) PSSA-N+R4 doped PAN-MMT composites, (f) de-doped PSSA-N+R4-PAN-MMT composites.

### Example-19

### PAN-MMT-PS Blends Foaming Process

The PAN-MMT-PS polymer pallets, used for the XRD study, were also used to form foams in a batch foaming apparatus, where the polymer pallets were placed on separated metal trays inside the high pressure CO₂ chamber for foaming. The PAN-MMT-PS blended pallets were about 30 mm in diameter and 3 mm in thickness. The optimized operation condition of foaming was at 120° C. under a high pressure of 2000 psi for ˜24 hours. The pressure drop rate was less than 5 seconds from 2000 psi to ambient conditions. It was found that the foam morphology was highly related to the dopants, doped and de-doped foams in PAN-MMT composites. The SEM morphology images at ×100 magnification given in FIGS. 11 and 12 show a common phenomenon that the foams from “de-doped EB form”, PAN EB-MMT-PS blends were well developed compared to the “doped ES form” blends, PAN ES-MMT-PS blends. The cell sizes were also varied by changing the dopants and the de-doping process. For example, the cell size of the de-doped EB form was much smaller than that of the doped form with the same dopant. These results are also confirmed by a density study and cell number density study on the same foams listed in Table 5. Table 5 summarizes the density and cell number density of PAN-MMT-PS foams at both doped ES-form and de-doped EB-form. We can conclude that the foam density of de-doped EB-forms is significantly lower than that of the doped ES-forms. The cell density of de-doped EB-foam is significantly higher than that of the doped ES-foam. One explanation is the “week acidity” of the CO₂ blowing agent. The CO₂ acts as a “dopant” to coordinate with the “de-doped” EB-form resin, allowing more CO₂ to be associated with the blended resin and to stay longer during foaming. On the other hand, the “doped” ES-form resin does not coordinate with the acidic CO₂ blowing agent. In addition, both inorganic salt forms and organic salt forms are shown to coordinate with the CO₂ blowing agent. The optimization and selection of those compounds are important when CO₂ is used as a foam blowing agent. The role of the nanoclay (MMT) or graphite is to supply a dispersible and high surface area substrate to foam PAN-MMT or PAN-graphite nanocomposite. The role of polyaniline is to supply a functional substrate, which can allow various dopants and salts to associate with the nanocomposite and act as a molecular CO₂ reservoir to control the CO₂ foaming.

TABLE 5
Density and cell density of Pani-MMT-PS foams:

| | | | Ave. cell | |
|---|---|---|---|---|
| | | Density | diameter | Cell Density |
| Acid category | Do-ping | (g/cm³) | (micron meter) | (cell #/cm³) |
| DBSA-Acid | ES | 0.4025 | 230 | 9.7 × 10E4 |
| DBSA-NH₄ salt | EB | 0.0586 | 86.4 | 2.8 × 10E6 |
| PSSA-Acid | ES | 0.2842 | 52.3 | 9.9 × 10E6 |
| PSSA-NH₄ salt | EB | 0.0671 | 28.3 | 7.8 × 10E7 |
| pTSA-Acid | ES | 0.4844 | 125.6 | 5.2 × 10E5 |
| pTSA-NH₄ salt | EB | 0.0308 | 48.0 | 7.4 × 10E7 |
| NH4—MgSO₄ salt | EB | 0.1073 | 80.9 | 1.6 × 10E7 |
| HCl—MgSO₄ acid | ES | 0.1150 | 48.1 | 3.2 × 10E6 |
| HCl-Acid | ES | 0.1180 | 140.3 | 6.1 × 10E5 |
| NH₄Cl salt | ES | 0.0665 | 98.4 | 1.9 × 10E6 |
| NR₄Cl—HCl acid | ES | 0.1557 | 181 | 2.7 × 10E5 |
| NR₄Cl—NH₄ salt | EB | 0.0506 | 38.2 | 3.1 × 10E7 |

Summarized, novel functionalized polyaniline was synthesized through in-situ polymerization with various dopants and particles. The material composition and CO₂ sequestration were investigated through various analytical techniques, such as, X-Ray diffraction (XRD), SEM, TEM, dielectric measurement, UV/vis, Rubotherm high pressure/vacuum absorption, XPS, elemental analysis, and FTIR. Those functional conducting polymers and their composite particles have superior capabilities to sequestrate not only CO₂ but also SOₓ and NOₓ at low temperatures. Studies were also conducted to demonstrate the conversion of the sequestrated CO₂ into fertilizers such as NH₄HCO₃, NH₄HSO_{4,} and NH₄NO₃ at ambient conditions. The reaction yields, product purity, thermal dynamic and kinetic of the reaction were also investigated. The performance of our materials is better than the best exiting amine-based CO₂ sorbent. The new polyaniline composite particles and associated processing techniques may lead to a low-cost CO₂ sequestration process in the industry that can permanently remove CO₂ from the emitting sources.

Due to environmental concerns of the currently used blowing agents—chlorofluorocarbons (HCFCs) and fluorocarbons (HFCs)—CO₂ has been identified as one of the most promising foaming agents for polymer foams because it is nonflammable, inexpensive, nontoxic and environmentally friendly. However, CO₂ also has its drawbacks of low solubility and high diffusivity in polymers compared to existing blowing agents. This often results in inferior foam density and cell morphology. As a result, the foam insulation performance is often low and inconsistent. The new polyaniline composite particles can be also blended with various polymers such as polystyrene, PMMA, PVC to generate polymer foam by utilizing CO2 as a blowing agent. It was found that the acid doped and salt de-doped polyaniline composite would result in different cell morphologies. As an example, the acid doped polyaniline could generate open cells, which should be significantly interested in acoustic insulation, filtration applications. However, the salt de-doped polyaniline composite will generate small cell size and low foam density, which should be interested in thermal insulation applications. The mixing of above two types of polyaniline will results in a bi-model cell and tri-model cell morphology, which has a significant impact on mechanical and electric performance such as, tensile strength, modulus, compact resistance, and dielectric constants and loss factor. Therefore, Pani-particle nanocomposites could act as a “molecular CO₂ reservoir” to adsorb and control CO₂ release during foaming, allowing cost effective replacement of CFC/HCFC/HFC blowing agents.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A CO₂ reservoir, comprising:
a functional conducting polymer; and
a plurality of particles coated with the functional conducting polymer, wherein the particles comprise nanoscale or microscale particles and their mixture.

2. The CO₂ reservoir according to claim 1, wherein the functional conducting polymer comprises CO₂ affinity group and NH₃ affinity group.

3. The CO₂ reservoir according to claim 1, wherein the functional conducting polymer comprises one or any combination selected from the group consisting of polyaniline, polypyrrole, polythiophene, polyphenylene vinylene, polyphenylene and their derivatives.

4. The CO₂ reservoir according to claim 1, wherein the functional conducting polymer is 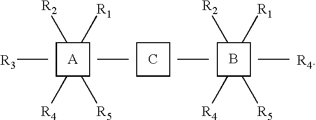
wherein A is selected from one of the following: —H, —CH₂—, —CH₂—CH₂—, —(CH₂)ₓ—, —C₆H₆—, C₆H₁₀—, —C₁₀H₆—, —C₁₄H₈—, —C₁₈H₁₂—, —CH═CH—, —C═C—, wherein B is selected from one of the following: —H, —CH₂—, —CH₂—CH₂—, —(CH₂)ₓ—, —C₆H₆—, C₆H₁₀—, —C₁₀H₆—, —C₁₄H₈—, —C₁₈H₁₂—, —CH═CH—, —C═C—, wherein, the binding group C is selected from one of the following group: —CH₂—, —CH₂—CH₂—, —(CH₂)ₓ—, —C₆H₆—, —C₆H₄—, —C₆H₁₀—, —C₁₀H₆—, —C₁₄H₈—, —C₁₈H₁₂—, —CH═CH—, —C═C—, —NH—, —N═, —O—, —CO—, —COO—, —CONH—, —S—, —SO—, —SO₂—, —PO₂—, —BH—, —B(OH)—, wherein R₁˜R₂ are individually selected from one of the following CO₂ affinity groups: —NH₂—, —(R)ₓ—NH₂— wherein R is —(CH₂)ₓ—, —(CH₂)ₓ—CO—NH₂, —B(OH)₂, —(CH₂)ₓ—C₂H₂O, wherein R₄˜R₅ are individually selected from one of the following NH₃ affinity groups: —COOH, —(R)ₓ—COOH wherein R is —(CH₂)ₓ—, —(CH₂)ₓ—COOR wherein R is —(CH₂)ₓ—, —SO₃H—, wherein R₃ and R₆ are individually selected from one of the following groups: H, —CH₃, -Et, -Bu, —COOH, —OH, —NH₂, aniline, sulfonated aniline, OH-aniline, COOH-aniline.

5. The CO₂ reservoir according to claim 1, wherein the functional conducting polymer is 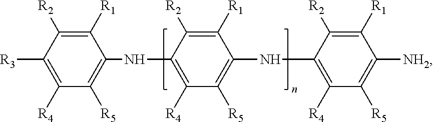
and n is an integer equal or more than 1, wherein R₁˜R₂ are individually selected from one of the following CO₂ affinity groups: H, —NH₂—, —(R)ₓ—NH₂— wherein R is —(CH₂)ₓ—, —(CH₂)ₓ—CO—NH₂, —B(OH)₂, —(CH₂)ₓ—C₂H₂O, wherein R₄˜R₆ are individually selected from one of the following NH₃ affinity groups: H, —COOH, —(R)ₓ—COOH wherein R is —(CH₂)ₓ—, —(CH₂)ₓ—COOR wherein R is —(CH₂)ₓ—, —SO₃H—, wherein R3 is selected from one of the following groups: H, —CH₃, -Et, -Bu, —NH₂, aniline, sulfonated aniline, OH-aniline, COOH-aniline.

6. The CO₂ reservoir according to claim 1, wherein the functional conducting polymer is 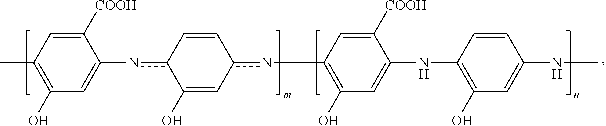
wherein n is an integer equal or more than 1, and m is an integer equal or more than 1.

7. The CO₂ reservoir according to claim 1, wherein the functional conducting polymer is 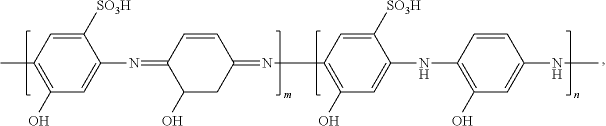
wherein n is an integer equal or more than 1, and m is an integer equal or more than 1.

8. The CO₂ reservoir according to claim 1, wherein the functional conducting polymer is 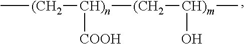
wherein n is an integer equal or more than 1, and m is an integer equal or more than 1.

9. The CO₂ reservoir according to claim 1, wherein the functional conducting polymer is 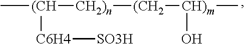
wherein n is an integer equal or more than 1, and m is an integer equal or more than 1.

10. The CO₂ reservoir according to claim 1, further comprising
an associating compound, wherein the associating compound can associate with the functional conducting polymer to form a composite, wherein the associating compound is selected from one of the following types: “p-type” associating compounds supplying “positive charges” to associate the composite, “n-type” associating compounds supplying “negative charges” to associate the composite, protonation associating compounds supplying “protonation” to associate the composite, polymeric associating compounds supplying “polymeric charges” to associate the composite, ionic liquid associating compounds supplying ionic liquid to associate the composite, surfactant associating compounds supplying surfactants to associate the composite, salt associating compounds supplying salt to associate the composite, hydrogen-bonding associating compounds supplying hydrogen bonding to associate the composite.

11. The CO₂ reservoir according to claim 10, wherein the particles supply surface and porous areas for the functional conducting polymer and the associating compound, wherein the particles are selected from one of the following type: inorganic particle, organic particle, non-reacted particle not reacting with CO₂, and reacted particle reacting with CO₂ or generate CO₂.

12. The CO₂ reservoir according to claim 11, wherein the particles are selected from at least one of the following: clay, silica, alumina, TiO₂, Talc, Boron Nitride (BN), graphite, graphene, carbon nanotubes, carbon nanofibers, active carbons, carbon woods, carbon-black, carbon fiber, glass fiber, glass beads, zeolite, polymeric beads, and polymeric particles.

13. The CO₂ reservoir according to claim 10, wherein the amount of each individual component is variable from the ranges of 0.02 wt. % to 99.95 wt. %, and is added up to a total of 100%.

14. The CO₂ reservoir according to claim 10, wherein the range of the functional conducting polymer is from 20 wt. % to 90 wt. %, the range of the associating compound is from 20 wt. % to 90 wt. %, and the range of the particle is from 5 wt. % to 80 wt. %.

15. The CO₂ reservoir according to claim 10, wherein the composite is preparation by one of the following method or the mix thereof: in-situ polymerization of the functional conducting polymer with the associating compound on the particles, electrically synthesizing the functional conducting polymer with the associating compound on the particles, solution coating the functional conducting polymer with the associate compound on the particles, spray coating the functional conducting polymer with associate the associating compound on the particles, solution blending the functional conducting polymer with the associate compound on the particles, extrusion blending the functional conducting polymer with the associating compound on the particles, vapor phase polymerization of coating the functional associating polymer with the associating compound on the particles.

16. The CO₂ reservoir according to claim 10, wherein the backbone of the functional associating polymer comprises at least a side group —R, wherein R is selected from one of the following: H, —CH₃, —OCH₃, -Et, -Bu, —CH₂—CH₂—NH₂, —(CH₂)ₙ—NH₂, —(CH₂)ₙ—OH, —COOH, —SO₃H—, —B(OH)₂, —OH, as well as a polymeric chains, wherein the functional associating polymer has a function to chemically associate the CO₂ when R is selected from the amine based chains, wherein the functional associating polymer has a function to physically associate with CO₂ when R is selected from the hydroxyl (—OH) based chains, wherein the functional associating polymer has NH₃ affinity function when R is selected from acidic group (—COOH, —SO₃H—), wherein the functional associating polymer becomes a branched conducting polymer with high CO₂ affiliation groups on both chemical and physical association than the corresponding linear polymer when R is a polymer or conducting polymer.

17. The CO₂ reservoir according to claim 10, wherein the composite is used as a CO₂ collecting material to remove CO₂ from air and the CO₂ absorption amount is in the range of 1.0-6.0 m mol CO₂/g composite, and condense it into dry ice or react CO₂ with other species to form industrial products and/or fertilizers such as CaCO₃, Ca(HCO₃)₂, H₂CO₃, NH₄HCO₃, (NH₄)₂CO₃, KHCO₃, K₂CO₃, etc.

18. The CO₂ reservoir according to claim 17, wherein the composite reacts with other miner and volcanic ash materials such as Ca-phosphate (Ca₅(PO₄)₃F), Apatite (Ca₅(PO₄)₃F), Ca-silicate (CaAl2Si₂O₈), Feldspar Episodes (CaAl₂Si₂O₈), etc. to form fertilizers, kaolinite (Al₂Si₂O₅(OH)₄), Calcite (CaCO₃).

19. The CO₂ reservoir according to claim 10, wherein the composite will blend and mix with at least one polymer material to form a blended material, and the polymer material comprises polymers or ceramic precursors comprising one or any combination selected from the group consisting of thermoplastic polymers such as, PS, PVC, PVA, PET, PP, PE, PC, PET, PEN, nylon, PMMA, PAI, PEEK, liquid crystal polymer, TPO, PA, PLA, PCL, etc.; thermoset polymers such as, PU, Epoxy, PI, PA, Unsaturated polyester, Vinyl ester, Phenolic, etc.; and ceramic precursors such as silazane lithium salt and titanium tetrachloride (TiCl4), polyureasilazane ceramic precursor, aluminum-containing polycarbosilane, polyaluminocarbosilane, Boron-modied polysilylcarbodi-imides precursors, etc.

20. The CO₂ reservoir according to claim 19, wherein the blended material can mix with some blowing agents and co-blowing agents for foaming such as CO₂, N₂, hydrofluorocarbon, fluorocarbon, water, or mixtures thereof. Fluorocarbon and hydrofluorocarbon include CFC11, HCFC 123, HCFC 141b, and commercial products such as Arkema Forane® 134a, R-134a, HFC-134a, DuPont's Dymel® 134a, 152a, etc.

21. The CO₂ reservoir according to claim 19, wherein the blended material can form both closed-cell and open-cell structures and bimodal structure when using the particles or the composite.

22. The CO₂ reservoir according to claim 19, wherein the blended material can form lighter color foam when using the composite comprising polyaniline, surfactant associating compounds, and dispersed grapheme.

23. The CO₂ reservoir according to claim 19, wherein the blended material is a blended resin being used in extrusion foaming and injection molding processes, where water and CO₂ will be released to assist the foaming during the extrusion and injection molding.

24. CO₂ reservoir according to claim 10, wherein the composite is used as a gas collecting material such as, SOₓ, NOₓ, H₂S, from the combustion air and condense it into industrial products and/or fertilizers such as NH₄HSO₄, NH₄NO₃, etc.

25. CO₂ reservoir according to claim 10, wherein the particles or the composite can form nanopapers or nanopapers on a fiber veil or woven and non-woven fabric substrate for coating, filtration and membrane applications.

26. A method of CO₂ capture and conversion comprising:
loop I: absorbing CO₂ by the CO₂ reservoir of claim 1 to form doped FPAN—HCO₃₋; and
loop II: absorbing NH₃/H₂O by the FPAN—HCO₃₋ to form de-doped FPAN—NH₄₊.

27. The method according to claim 26, wherein, in loop I, the liquid or solid FPAN sorbent absorbs CO₂ in the sequestration reactor to form the doped FPAN—HCO₃₋ at low temperature (<90° C.) and low pressure (<10 psia) with a relatively high speed where the FPAN main chain chemically absorbs the CO₂ and OH— side chains physically absorb CO₂.

28. The method according to claim 26, wherein, in loop II, the doped FPAN—HCO₃₋ absorbs NH₃/H₂O in the fertilizer reactor to form de-doped FPAN—NH₄₊ at low temperature (<90° C.) and low pressure (<10 psia) with a high reaction rate (in seconds) where the —SO₃H— side chains chemically absorb the NH₃/H₂O and OH— side chains physically absorb NH₃/H₂O, and the FPAN—NH₄₊ quickly forms FPAN—NH₄₊/HCO₃₋ to release NH₄HCO₃ fertilizer at low temperature (<60° C.) and low pressure (<10 psia).
